(19) United States
(12) Patent Application Publication
(10) Pub. No.: US 2012/0225053 A1
(43) Pub. Date: Sep. 6, 2012

(54) COMPOSITIONS AND METHODS FOR THE PREVENTION AND TREATMENT OF CONDITIONS ASSOCIATED WITH INFLAMATION

(76) Inventors: Slavik Dushenkov, Fort Lee, NJ (US); Patricia Lucas-Schnarre, Metuchen, NJ (US); Julie Beth Hirsch, Branchburg, NJ (US); David Evans, Belle Mead, NY (US); Kitty Evans, Merritt Island, FL (US)

(21) Appl. No.: 11/920,973

(22) PCT Filed: May 24, 2006
(86) PCT No.: PCT/US06/20542

§ 371 Date: May 2, 2012

Related U.S. Application Data
(60) Provisional application No. 60/684,487, filed on May 24, 2005.

(51) Int. Cl. A61K 31/353; A61P 11/06; A61P 25/00; A61P 1/00; A61P 11/00; A61P 37/08; A61P 31/00; A61P 19/02; A61P 17/06; A61K 31/7008; A61K 38/48; A61K 36/287; A61K 31/715; A61K 38/39; A61K 35/60; A61K 36/906; A61K 36/16; A61K 36/258; A61K 36/87; A61K 36/76; A61K 36/736; A61K 36/81; A61P 29/00

(52) U.S. Cl. 424/94.65; 514/456; 514/62; 424/771; 514/54; 514/17.2; 424/523; 424/756; 424/752; 424/728; 424/766; 424/769; 424/735; 424/760

(57) ABSTRACT

The present invention provides methods for preventing, treating, managing and/or ameliorating a condition associated with inflammation (e.g., an inflammatory disorder) or a symptom thereof, the methods comprising administering to a subject in need thereof an effective amount of a theaflavin composition and an effective amount of one or more therapies other than such a theaflavin composition. In particular, the present invention provides methods for preventing, treating, managing and/or ameliorating a condition associated with inflammation (e.g., an inflammatory disorder) or a symptom thereof, the methods comprising administering to a subject in need thereof an effective amount of a theaflavin composition, an effective amount of a glucosamine composition, and optionally one or more other therapies.

This application is entitled to and claims priority benefit under 35 U.S.C. §119(e) to U.S. Provisional application Ser. No. 60/684,487 filed May 24, 2005, which is incorporated herein by reference in its entirety.

### FIELD OF THE INVENTION

The present invention provides methods for preventing, treating, managing and/or ameliorating a condition associated with inflammation (e.g., an inflammatory disorder) or a symptom thereof, the methods comprising administering to a subject in need thereof an effective amount of a theaflavin composition and an effective amount of one or more therapies other than such a theaflavin composition.

### BACKGROUND OF THE INVENTION

Inflammatory Disorders

Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events can result in swelling, redness, warmth (altered heat patterns), and pus formation at the site of injury or infection.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction.

Diseases such as rheumatoid arthritis, osteoarthritis, Crohn's disease, asthma, allergies and inflammatory bowel disease, are characterized by chronic inflammation. Current treatments for inflammatory disorders involve symptomatic medications and immunosuppressive agents to control symptoms. For example, nonsteroidal anti-inflammatory drugs (NSAIDs) such as aspirin, ibuprofen, fenoprofen, naproxen, tolmetin, sulindac, meclofenamate sodium, piroxicam, flurbiprofen, diclofenac, oxaprozin, nabumetone, etodolac, and ketoprofen have analgesic and anti-inflammatory effects. However, NSAIDs are believed not to be capable of altering progression of the disease. (Tierney et al. (eds), Current Medical Diagnosis & Treatment, 37 ed., Appleton & Lange (1998), p793). Moreover, NSAIDs are known to cause gastrointestinal side effects. Corticosteroids are another class of drugs that are commonly used to control inflammatory symptoms. Corticosteroids, like NSAIDs, do not alter the natural progression of the disease, and thus, clinical manifestations of active disease commonly reappear when the drug is discontinued. Low doses of immunosuppressive agents such as cytotoxic agents are also commonly used in the treatment of inflammatory disorders. Many cytotoxic agents frequently cause stomatitis, erythema, slopecia, nausea, vomiting, diarrhea, and damage to major organs such as the kidney and liver. Further, the long-term usage of immunosuppressive agents usually leaves the patient defenseless to infections.

New treatment and preventative modalities for inflammatory disorders are constantly being sought. In particular, any new modality that reduces the dosage and/or frequency of administration of agents currently being used, or is capable of making a currently used treatment and/or prevention more effective is constantly being sought.

Theaflavins

Black tea extracts have been reported to have a protective and therapeutic effect for a number of disorders, including cancer and inflammatory conditions. The major polyphenols characteristic of black tea are: theaflavin (TF-1), theaflavin-3-gallate and theaflavin-3′-gallate mixture (TF-2), and theaflavin-3,3′-digallate (TF-3). These theaflavin polyphenols are fermentation products derived from green tea polyphenols and are responsible for the characteristic color, fragrance and taste of black tea.

Citation of any reference in Section 2 of this application is not to be construed as an admission that such reference is prior art to the present application.

### SUMMARY OF THE INVENTION

In one aspect, the present invention provides methods and compositions relating to combinations of anti-inflammatory therapies. For example, the invention provides, in one embodiment, methods for preventing, treating, managing and/or ameliorating a symptom or a condition associated with inflammation, the methods comprising administering to a subject in need thereof a composition comprising theaflavin (TF), theaflavin-3-gallate (TF-3-G), theaflavin 3′-gallate (TF-3′-G) and/or theaflavin-3,3′-digallate (TF:-3,3′-diG), including black tea extracts, and certain anti-inflammatory therapies. In another embodiment, the present invention provides methods for preventing, treating, managing and/or ameliorating a symptom or a condition associated with inflammation, the methods comprise administering to a subject in need thereof a composition comprising TF, TF-3-G, TF-3′-G and/or TF-3,3-diG, including black tea extracts, and a therapy that slows cartilage degradation and/or rebuilds cartilage, such as, e.g., glucosamine. The invention encompasses dietary, prophylactic or therapeutic protocols for preventing, treating, managing and/or ameliorating conditions associated with inflammation.

The present invention provides methods for relieving a symptom of inflammation in a subject, such as but not limited to redness, swelling, edema, excess warmth and pain, the methods comprising administering to a subject in need thereof an effective amount of a theaflavin composition and an effective amount of one or more therapies other than such a theaflavin composition (e.g., a glucosamine composition). In a specific embodiment, the present invention provides methods for reducing the redness, swelling, edema, excess warmth and/or pain associated with inflammation, by 20% (in some embodiments, 25%, 30%, 35%, 40%, 45%, 50%, 55%, 60%, 65%, 70%, 75%, 80%, 85%, 90%, 95% or more) in a subject, the methods comprising administering to a subject in need thereof an effective amount of a theaflavin composition and an effective amount of one or more therapies other than such a theaflavin composition (e.g., a glucosamine composition) as determined by methods well-known in the art.

The present invention also provides methods for preventing, treating, managing and/or ameliorating of a condition associated with inflammation (e.g., an inflammatory disorder) or a symptom thereof, the methods comprising administering to a subject in need thereof an effective amount of a theaflavin composition and an effective amount of one or more therapies other than such a theaflavin composition (e.g., a glucosamine composition). Administration of such therapies can, for example, be via one or more compositions, food additives, dietary supplements, nutraceutical compositions or food compositions of the invention. In certain embodiments, the condition associated with inflammation is an acute condition. In other embodiments, the condition associated with inflammation is a chronic condition. Examples of inflammatory disorders that can be prevented, treated, managed and/or ameliorated in accordance with the invention include, but are not limited to, asthma, encephilitis, inflammatory bowel disease, chronic obstructive pulmonary disease (COPD), allergic disorders, septic shock, fibrosis (including, e.g., pulmonary fibrosis), undifferentitated spondyloarthropathy, undifferentiated arthropathy, arthritis, juvenile arthritis, psoriatic arthritis, rheumatoid arthritis, osteoarthritis, psoriasis, inflammatory osteolysis, degenerative joint diseases, and chronic inflammation resulting from e.g., chronic viral or bacteria infections.

The invention also provides methods for preventing, treating, managing and/or ameliorating an adverse health condition associated with the activation of nuclear factor-kappa B (“NF-κB”), the nuclear translocation of NF-κB, the binding of NF-κB to DNA in cells, and/or COX-2 gene expression, the methods comprising administering to a subject in need thereof an effective amount of a theaflavin composition and an effective amount of one or more therapies other than such a theaflavin composition. Administration of such therapies can, for example, be via one or more compositions, food additives, dietary supplements, nutraceutical compositions or food compositions of the invention. Examples of an adverse health condition associated with the activation of NF-KB, the nuclear translocation of NF-κB, the binding of NF-κ3 to DNA in cells, and/or Cox-2 gene expression include, but are not limited to, an inflammatory disorder, or any symptoms associated with such disorders.

Non-limiting examples of therapies that can be administered in combination with a theaflavin composition include anti-viral agents, antibiotic agents, TNF-α antagonists, immunomodulatory agents, anti-cancer agents, and anti-inflammatory agents. In a preferred embodiment, a theaflavin composition is administered in combination with a glucosamine composition to a subject with a condition associated with inflammation (e.g., an inflammatory disorder). In certain embodiments, a theaflavin composition of the invention is not administered in combination with a rosemary extract, a Mexican Bamboo extract, a Huzhang extract, resveratrol, a hydroxylated resveratrol analog, a methoxylated resveratrol analog, a green tea extract, an orange peel extract, a polymethoxylated flavone or a hydroxy-polymethoxyflavone found in orange peel extract.

In a specific embodiment, the administration of a theaflavin composition in combination with a therapy other than such a theaflavin composition to a subject with a condition associated with inflammation (e.g., an inflammatory disorder) produces a better prophylactic or therapeutic effect in the subject than either therapy alone. In certain embodiments, the administration of a theaflavin composition in combination with a therapy other than such a theaflavin composition to a subject with a condition associated with inflammation (e.g., an inflammatory disorder) achieves a 1.5 fold, preferably a 2 fold, 3 fold, 4 fold, 5 fold, 6 fold, 7 fold, 8 fold, 9 fold, 10 fold, 15 fold or 20 fold better prophylactic or therapeutic effect in the subject than either therapy alone. In other embodiments, the administration of a theaflavin composition in combination with a therapy other than such theaflavin composition to a subject with a condition associated with inflammation (e.g., an inflammatory disorder) achieves a 10%, preferably 15%, 20%, 25%, 30%, 35%, 40%, 45%, 50%, 55%, 60%, 65%, 70%, 75%, 80%, 85%, 90%, 95%, 100%, 125%, 150%, or 200% better prophylactic or therapeutic effect in the subject than either therapy alone. In particular embodiments, the administration of a theaflavin composition in combination with a therapy other than such a theaflavin composition to a subject with a condition associated with inflammation (e.g., an inflammatory disorder) achieves a 20%, preferably a 25%, 30%, 35%, 40%, 45%, 50%, 55%, 60%, 65%, 70%, 75%, 80%, 85%, 90%, 95% or 98% greater reduction in the inflammation of a particular organ, tissue or joint in the subject than either therapy alone. In other embodiments, the administration of a theaflavin composition in combination with a therapy other than such a theaflavin composition to a subject with a condition associated with inflammation (e.g., an inflammatory disorder) has an a more than additive effect or synergistic effect in the subject.

In certain embodiments, the administration of a theaflavin composition in combination with a therapy other than such a theaflavin composition to a subject with a condition associated with inflammation (e.g., an inflammatory disorder) achieves a 1.5 folded, preferably 2 fold, 3 fold, 4 fold, 5 fold or greater reduction in the activation of NF-κB, the nuclear translocation of NK-κB, the binding of NK-κB to DNA, and/or Cox-2 gene expression in cells contacted with the combination relative to cells not contacted with the combination or negative control cells (e.g., cells contacted with phosphate buffered saline (PBS)) under the same conditions. In other embodiments, the administration of a theaflavin composition in combination with a therapy other than such a theaflavin composition to a subject with a condition associated with inflammation (e.g., an inflammatory disorder) achieves a 20%, preferably 25%, 30%, 35%, 40%, 45%, 50%, 55%, 60%, 65%, 70%, 75%, 80%, 85%, 90% or 95% reduction in the activation of NF-κB, the nuclear translocation of NK-κB, the binding of NK-KB to DNA, and/or Cox-2 gene expression in cells contacted with the combination relative to cells not contacted with the combination or negative control cells (e.g., cells contacted with phosphate buffered saline (PBS)) under the same conditions. In a specific embodiment, the cells are from a subject with a condition associated with inflammation. In some embodiments, the condition is an acute condition. In other embodiments, the condition is a chronic condition.

In certain embodiments, the methods of the invention enable lower dosages of a theaflavin composition and/or less frequent administration of a theaflavin composition to a subject with a condition associated with inflammation (e.g., an inflammatory disorder) to achieve a prophylactic or therapeutic effect. In other embodiments, the methods of the invention enable lower dosages of the therapies utilized in combination with a theaflavin composition for the prevention, treatment, management and/or amelioration of a condition associated with inflammation (e.g., an inflammatory disorder) and/or less frequent administration of such therapies to a subject with a condition associated with inflammation to achieve a prophylactic or therapeutic effect. In yet other embodiments, the methods of the invention reduce or avoid unwanted or adverse side effects associated with the administration of current single agent therapies and/or existing combination therapies for a condition associated with inflammation (e.g., an inflammatory disorder), which in turn improves patient compliance with the dietary, prophylactic or therapeutic protocol.

In one embodiment, a theaflavin composition comprises one, two, three or all of the following theaflavins: TF, TF-3-G, TF-3′-G and/or TF-3,3′-diG, or pharmaceutically acceptable salts, solvates or hydrates thereof. In another embodiment, a theaflavin composition comprises any combination of the following theaflavins: TF, TF-3-G, TF-3′-G and/or TF-3,3′-diG, or pharmaceutically acceptable salts, solvates or hydrates thereof. In another embodiment, a theaflavin composition or is from comprises TF, TF-3-G, TF-3′-G and TF-3,3′-diG, or pharmaceutically acceptable salts, solvates or hydrates thereof. In another embodiment, a theaflavin composition is or is from a natural source of theaflavins (e.g., TF, TF-3-G, TF-3′-G and TF-3,3′-diG). In another, alternative embodiment, a theaflavin composition is not or is not from a natural source of theaflavins (e.g., TF, TF-3-G, TF-3′-G and TF-3,3′-diG). In another embodiment, a theaflavin composition is a black tea extract. In another, alternative embodiment, a theaflavin composition is not a black tea extract.

In one aspect, a theaflavin composition of the invention comprises: (i) a concentration of a theaflavin(s) (e.g., TF, TF-3-G, TF-3′-G and/or TF-3,3′-diG) that is different from that in a natural source of theaflavins; and/or (ii) the ratio of the concentration of one of the theaflavins (e.g., TF, TF-3-G, TF-3′-G and/or TF-3,3′-diG) in the therapy to that of another theaflavin in the therapy is different from that in a natural source of the theaflavins. Such a theaflavin composition of the invention can be prepared, for example, by processing a natural source of theaflavins (e.g., black tea extract) such that at least one particular theaflavin (e.g., TF, TF-3-G, TF-3′-G and/or TF-3,3′-diG) has been selectively removed, retained, or enriched. Alternatively, one or more purified theaflavins (e.g., TF, TF-3-G, TF-3′-G and/or TF-3,3′-diG) can be used to make such therapies. Such a composition can also be prepared, for example, by adding an amount of at least one theaflavin (e.g., TF, TF-3-G, TF-3′-G and/or TF-3,3′-diG) to a natural source or processed form of a natural source of the theaflavins.

In various embodiments, a theaflavin composition of the invention comprises a mixture of TF, TF-3-G, TF-3′-G and/or TF-3,3′-diG, wherein the concentration of one or more of the theaflavins is increased or decreased relative to that in a natural source of the theaflavins. In specific embodiments, a theaflavin composition of the invention comprises a mixture of TF, TF-3-G, TF-3′-G and/or TF-3,3′-diG, wherein the ratio of the concentration of one of the theaflavins in the therapy to that of another theaflavin in the therapy is increased or decreased relative to the ratio of the concentration of the same theaflavins in a natural source of the theaflavins. In other embodiments, the invention provides a theaflavin composition comprising a mixture of TF, TF-3-G, TF-3′-G and/or TF-3,3′-diG, or pharmaceutically acceptable salts, solvates or hydrates thereof, wherein the percentages (by dry weight) of one or more theaflavins relative to the total content of theaflavins in the therapy is different from that in a natural source of the theaflavins. In yet other embodiments, a theaflavin composition of the invention comprises a mixture of TF, TF-3-G, TF-3′-G and/or TF-3,3′-diG, or pharmaceutically acceptable salts, solvates or hydrates thereof, wherein the ratio of certain theaflavins in the therapy is different from that in a natural source of theaflavins.

In certain embodiments, a theaflavin composition comprises a theaflavin component and a non-theaflavin component. In certain embodiments, the non-theaflavin component comprises a pharmaceutically acceptable carrier, vehicle or excipient. In certain embodiments, the non-theaflavin component comprises an active ingredient such as a compound with anti-inflammatory activity. In other embodiments, the non-theaflavin component does not comprise a compound with anti-inflammatory activity as detectable in an in vitro assay and/or an animal model for inflammation. In a specific embodiment, the non-theaflavin component does not have anti-inflammatory activity as measured by the inhibition of carrageenan-induced paw edema in the rat, as described infra and known in the art. In certain embodiments, the theaflavin component consists of or consists essentially of one, two, three or all of the following theaflavins: TF, TF-3-G, TF-3′-G and/or TF-3,3′-diG, or pharmaceutically acceptable salts, solvates or hydrates thereof.

In certain embodiments, a theaflavin composition consists of or consists essentially of a theaflavin(s)of the invention, and a pharmaceutically acceptable carrier, vehicle or excipient. In other embodiments, a theaflavin composition comprises a theaflavin(s) of the invention, as the active ingredient, and a pharmaceutically acceptable carrier, vehicle or excipient. In accordance with these embodiments, the theaflavin composition does not comprise active ingredients other than the theaflavins described herein. In certain embodiments, a theaflavin composition does not comprise glucosamine.

The present invention provides glucosamine compositions comprising glucosamine, or a pharmaceutically acceptable salt, solvate or hydrate thereof, which can be utilized in combination with a theaflavin composition of the invention to prevent, treat, manage and/or ameliorate a condition associated with inflammation (e.g., an inflammatory disorder) or a symptom thereof. Non-limiting examples of salt forms of glucosamine include glucosamine sulfate, glucosamine hydrochloride, and n-acetyl glucosamine. In one embodiment, a glucosamine composition is or is from a natural source of glucosamine. In another, alternative embodiment, a glucosamine composition is not or is not from a natural source of glucosamine.

In certain embodiments, a glucosamine composition consists of or consists essentially of glucosamine, or a pharmaceutically acceptable salt, solvate or hydrate thereof. In a specific embodiment, a glucosamine composition is composed of glucosamine, or a pharmaceutically acceptable salt, solvate or hydrate thereof, and a pharmaceutically acceptable carrier, vehicle or excipient.

In a preferred embodiment, an effective amount of a theaflavin composition and an effective amount of a glucosamine composition are administered to a subject to prevent, treat, manage and/or ameliorate a degenerative joint disease such as osteoarthritis. In certain embodiments, an effective amount of a theaflavin composition, an effective amount of a glucosamine composition and an effective amount of one or more other therapies are administered in to a subject to prevent, treat, manage and/or ameliorate a degenerative joint disease such as osteoarthritis.

The present invention provides compositions comprising theaflavin, theaflavin-3-gallate, theaflavin-3′-gallate, theaflavin-3,3′-digallate, or a pharmaceutically acceptable salt, solvate or hydrate thereof and glucosamine (e.g., glucosamine sulfate, glucosamine hydrochloride, n-acetyl glucosamine), or a pharmaceutically acceptable salt, solvate or hydrate thereof. Such compositions can be utilized to prevent, treat, manage and/or ameliorate a condition or symptom associated with inflammation (e.g., an inflammatory disorder).

The therapies of the present invention can be administered concomitantly or sequentially to a subject. The therapies of the present invention can also be cyclically administered. Cycling therapy involves the administration of a first therapy for a period of time, followed by the administration of a second therapy for a period of time and repeating this sequential administration, i.e., the cycle, in order to reduce the development of resistance to one of the therapies, to avoid or reduce the side effects of one of the therapies, and/or to improve the efficacy of the therapies.

The therapies of the present invention can be administered to a subject concurrently. The term “concurrently” is not limited to the administration of two or more therapies at exactly the same time, but rather it is meant that a theaflavin composition and the other therapy are administered to a subject in a sequence and within a time interval such that the theaflavin composition can act together with the other therapy to provide an increased benefit than if they were administered otherwise. For example, each therapy may be administered at the same time or sequentially in any order at different points in time; however, if not administered at the same time, they should be administered sufficiently close in time so as to provide the desired therapeutic or prophylactic effect. Each therapy can be administered separately, in any appropriate form and by any suitable route. In various embodiments, the therapies are administered less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more therapies are administered within the same patient visit.

The therapies of the invention (i.e., a theaflavin composition and at least one other therapy) can be administered to a subject in the same composition. Alternatively, the therapies can be administered concurrently to a subject in separate compositions. The therapies may be administered to a subject by the same or different routes of administration.

The present invention provides nutraceutical compositions comprising a theaflavin composition of the invention, and one or more prophylactic or therapeutic agents in addition to the theaflavin composition. In one embodiment, the nutraceutical compositions of the invention are prepared from natural sources. In an alternative embodiment, the nutraceutical compositions are not prepared or are not entirely prepared from natural sources. In a preferred embodiment, a nutraceutical composition comprises a theaflavin composition, a glucosamine composition, and optionally one or more other prophylactic or therapeutic agents.

The present invention also provides dietary supplements comprising a theaflavin composition of the invention, and one or more prophylactic or therapeutic agents in addition to the theaflavin composition. In one embodiment, the dietary supplements of the invention are prepared from natural sources. In an alternative embodiment, the dietary supplements of the invention are not prepared or are not entirely prepared from natural sources. In a preferred embodiment, a dietary supplement comprises a theaflavin composition, a glucosamine composition, and optionally one or more other prophylactic or therapeutic agents.

The present invention also provides food additives comprising a theaflavin composition of the invention, and one or more prophylactic or therapeutic agents in addition to the theaflavin composition. In one embodiment, the food additives of the invention are prepared from natural sources. In an alternative embodiment, the food additives of the invention are not prepared or are not entirely prepared from natural sources. In a preferred embodiment, a food additive comprises a theaflavin composition, a glucosamine composition, and optionally one or more other prophylactic or therapeutic agents.

The present invention also provides food compositions comprising a theaflavin composition of the invention, and one or more prophylactic or therapeutic agents in addition to the theaflavin composition. In one embodiment, the food compositions of the invention are prepared from natural sources. In an alternative embodiment, the food composition of the invention are not prepared or are not entirely prepared from natural sources. In a preferred embodiment, a food composition comprises a theaflavin composition, a glucosamine composition, and optionally one or more other prophylactic or therapeutic agents.

The present invention also provides pharmaceutical compositions comprising a theaflavin composition of the invention, and one or more prophylactic or therapeutic agents in addition to the theaflavin composition. In one embodiment, such prophylactic or therapeutic agents are known to be useful, or have been or are currently being used to the prevention, treatment, management and/or amelioration of an inflammatory disorder or a condition, or one or more symptoms thereof. In a preferred embodiment, a pharmaceutical composition comprises a theaflavin composition, a glucosamine composition, and optionally one or more other prophylactic or therapeutic agents.

In another aspect, the present invention provides articles of manufacture comprising, in one or more containers, a pharmaceutical composition, nutraceutical composition, dietary supplement, food additive or food composition of the invention.

Terminology and Abbreviations

As used herein, “a” or “an” means at least one, unless clearly indicated otherwise.

As used herein, the terms “about” or “approximately”, unless otherwise indicated, refer to a value that is no more than 10% above or below the value being modified by the term.

As used herein, the terms “antibody” and “antibodies” refer to molecules that contain an antigen binding site, e.g., immunoglobulins. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies include, but are not limited to, monoclonal antibodies, multispecific antibodies, human antibodies, humanized antibodies, camelised antibodies, chimeric antibodies, single domain antibodies, single chain Fvs (scFv), single chain antibodies, Fab fragments, F(ab') fragments, disulfide-linked Fvs (sdFv), and anti-idiotopic (anti-Id) antibodies (including, e.g., anti-Id antibodies to antibodies of the invention), and epitope-binding fragments of any of the above.

It is contemplated that, where the therapy(ies) of the invention occur in a natural source, the terms “composition” and “composition of the invention” are not intended to include a natural source of the therapy(ies) but can, in certain embodiments of the invention, encompass a physically and/or chemically modified form of the natural source. For example, if the therapy(ies) can be obtained from a plant, the terms are not intended to encompass the plant or an anatomical part of the plant (e.g., leaves of the plant), however, a solvent extract of the plant or plant part(s) can be a composition of the invention.

As used herein, the terms “purified” and “isolated” in the context of a compound or agent that is chemically synthesized refers to a compound or agent that is substantially free of chemical precursors or other chemicals when chemically synthesized. In a specific embodiment, the compound or agent is 60%, 65%, 75%, 80%, 85%, 90%, 95%, or 99% free (by dry weight) of other, different compounds or agents.

As used herein, the terms “purified” and “isolated” when used in the context of a compound or agent that can be obtained from a natural source, e.g., plants, refers to a compound or agent which is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials.

As used herein, the term “effective amount” generally refers to the amount of a therapy which is sufficient to reduce or ameliorate the severity, duration of a disorder or one or more symptoms thereof, prevent the advancement of a disorder, cause regression of a disorder, prevent the recurrence, development, or onset of a disorder or a symptom thereof, or enhance or improve the prophylactic or therapeutic effect(s) of another therapy.

As used herein, the term “therapeutically effective amount” refers to that amount of a therapy (e.g., a therapeutic agent) sufficient to result in the amelioration of one or more symptoms of a disorder, prevent advancement of a disorder, cause regression of a disorder, or to enhance or improve the therapeutic effect(s) of another therapy.

In a specific embodiment, an therapeutically effective amount refers to the amount of a therapy (e.g., a therapeutic agent) that reduces the inflammation of a organ or tissue (e.g., joint, skin, stomach lining). Preferably, a therapeutically effective of a therapy (e.g., a therapeutic agent) reduces the inflammation of a organ or tissue by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline. Examples of therapeutically effective amounts of therapies are provided infra.

As used herein, the phrase “prophylactically effective amount” refers to the amount of a therapy (e.g., prophylactic agent) which is sufficient to result in the prevention or inhbition of the development, recurrence or onset of a disorder or a symptom thereof, or to enhance or improve the prophylactic effect(s) of another therapy (e.g., another prophylactic agent). Examples of prophylactically effective amounts of therapies are provided infra.

As used herein, the term “in combination” refers to the use of more than one modalities (e.g., one or more prophylactic and/or therapeutic agents). The use of the term “in combination” does not restrict the order in which modalities are administered to a subject with a disorder. A first modality (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subse_{quent to} (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second modality (e.g., a prophylactic or therapeutic agent such as an anti-inflammatory agent) to a subject with a condition associated with inflammation (e.g., an inflammatory disorder) or a symptom thereof.

As used herein, the terms “manage,” “managing,” and “management” in the context of the administration of therapy to a subject refer to the beneficial effects that a subject derives from a therapy (e.g., a prophylactic or therapeutic agent), while not resulting in a cure of the condition associated with inflammation. In certain embodiments, a subject is administered one or more modalities (e.g., one or more prophylactic or therapeutic agents) to “manage” a condition associated with inflammation so as to prevent the progression or worsening of the condition.

As used herein, the terms “modality”, modalities”, “therapies” and “therapy” can refer to any protocol(s), method(s), composition(s), and/or agent(s) that can be used in the prevention, treatment, management, or amelioration of a disorder or one or more symptoms thereof. In certain embodiments, the terms “modality”, modalities”, “therapy” and “therapies” refer to chemotherapy, radiation therapy, surgery, hormonal therapy, biological therapy, immunotherapy and/or other therapies useful in the prevention, management, treatment or amelioration of a disorder or one or more symptoms thereof.

As used herein, the term “natural source” refers to a material that occurs in the natural environment, and may comprise one or more biological entities. For example, a natural source can be a plant, an animal, an anatomical part of a plant or animal, a microorganism, a mixture of different plants, animals, and/or microorganisms, or an environmental sample. It is not necessary that the biological entities present in a natural source be classified or characterized.

As used herein, the terms “non-responsive” and “refractory” describe patients treated with a currently available modality (e.g., a prophylactic or therapeutic agent) for a condition associated with inflammation, which is not clinically adequate to relieve one or more symptoms associated with such condition. Typically, such patients suffer from severe, persistently active disease and require additional therapy to ameliorate the symptoms associated with their condition.

As used herein, the phrase “pharmaceutically acceptable salt” refers to pharmaceutically acceptable organic or inorganic salts of a compound of the invention (e.g., TF, TF-3-G, TF-3′-G, TF-3,3′-diG or glucosamine). Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p toluenesulfonate, and pamoate (i.e., 1,1′-methylene bis (2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

As used herein, the term “pharmaceutically acceptable solvate” refers to an association of one or more solvent molecules and a compound of the invention (e.g., TF, TF-3-G, TF-3′-G, TF-3,3′-diG or glucosamine). Examples of solvents that form pharmaceutically acceptable solvates include, but are not limited to, water, isopropanol, ethanol, methanol, DMSO, ethyl acetate, acetic acid, and ethanolamine.

As used herein, the terms “prevent,” “preventing” and “prevention” in the context of the administration of a therapy to a subject refer to the prevention or inhibiting of the recurrence, onset, or development of a condition associated with inflammation or a symptom thereof in a subject resulting from the administration of a therapy (e.g., a prophylactic or therapeutic agent), or the administration of a combination of therapies (e.g., a combination of prophylactic or therapeutic agents).

As used herein, the terms “prophylactic agent” and “prophylactic agents” as used refer to any agent(s) which can be used in the prevention of a condition associated with inflammation or one or more symptoms thereof. In certain embodiments, the term “prophylactic agent” refers to a composition of the invention. In certain other embodiments, the term “prophylactic agent” does not refer to a composition of the invention. Prophylactic agents may be characterized as different agents based upon one or more effects that the agents have in vitro and/or in vivo.

As used herein, the phrase “side effects” encompasses unwanted, and adverse effects of a modality. Side effects are always unwanted, but unwanted effects are not necessarily adverse. An adverse effect might be harmful, uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra pyramidal symptoms, akathisia, cardiovascular disturbances and sexual dysfunction.

As used herein, the terms “subject” and “patient” are used interchangeably herein. The terms “subject” and “subjects” refer to an animal, preferably a mammal including a non-primate and a primate (e.g., a monkey such as a cynomolgous monkey, a chimpanzee, and a human), and more preferably a human. The term “animal” also includes, but is not limited to, companion animals such as cats and dogs; zoo animals; wild animals; farm animals such as ruminants, non-ruminants, livestock and fowl (e.g., horses, cattle, sheep, pigs, turkeys, ducks, and chickens); and laboratory animals, such as rodents (e.g., mice and rats), rabbits, and guinea pigs, as well as animals that are cloned or modified, either genetically or otherwise (e.g., transgenic animals).

In one embodiment, a subject in need of prevention, treatment, management, or amelioration of a condition associated with inflammation is a subject that has the condition, that is at risk of the condition, diagnosed with the condition, or that has recovered from a condition. In another embodiment, the subject is an animal, preferably a mammal, and more preferably a human, that is predisposed and/or at risk because of a genetic factor(s), an environmental factor(s), or a combination thereof to develop a condition associated with inflammation. In yet another embodiment, the subject is an immunocompromised or immunosuppressed mammal, such as a human. In an alternative embodiment, the subject is not an immunocompromised or immunosuppressed mammal, such as a human (e.g., an HIV patient). In yet another embodiment, the subject is refractory or non-responsive to current treatments for a condition associated with inflammation.

As used herein, the term “synergistic” refers to a combination of modalities (e.g., a composition of the invention comprising a theaflavin component and a non-theaflavin component (e.g., a prophylactic or therapeutic agent such as an anti-inflammatory compound), a combination of compositions of the invention and/or a combination of a compound, compounds or a composition of the invention and another modality (e.g., a prophylactic or therapeutic agent), including one which has been or is currently being used to prevent, manage or treat a disorder), which combination is more effective than the additive effects of the individual modalities. A synergistic effect of a combination of modalities (e.g., a combination of prophylactic or therapeutic agents) can permit the use of lower dosages of one or more of the modalities and/or less frequent administration of said modalities to a subject with a condition associated with inflammation. The ability to utilize lower dosages of a modality and/or to administer a modality less frequently can reduce the toxicity associated with the administration of the modality to a subject without reducing the efficacy of said agent in the prevention, management or treatment of a condition associated with inflammation. In addition, a synergistic effect can result in improved efficacy of modalities in the prevention, management and/or treatment of a condition associated with inflammation. Moreover, a synergistic effect of a combination of modalities can avoid or reduce adverse or unwanted side effects associated with the use of either modality alone.

As used herein, the terms “therapeutic agent” and “therapeutic agents” refer to any agent(s) which can be used in the treatment, management, or amelioration of a disorder or one or more symptoms thereof. In certain embodiments, the term “therapeutic agent” refers to a composition of the invention. In certain other embodiments, the term “therapeutic agent” does not refer to a composition of the invention. Therapeutic agents may be characterized as different agents based upon one or more effects the agents have in vivo and/or in vitro, for example, an anti-inflammatory agent may also be characterized as an immunomodulatory agent.

As used herein, the terms “treat”, “treatment” and “treating” in the context of the administration of a therapy to a subject refer to the reduction or amelioration of the progression, severity and/or duration of a condition associated with inflammation, or the amelioration of one or more symptoms thereof resulting from the administration of one or more modalities (e.g., a composition of the invention). In specific embodiments, such terms refer to a reduction in the swelling of one or more joints, organs or tissues, a reduction in redness of an inflammed area and/or a reduction in the pain associated with inflammation.

### DETAILED DESCRIPTION OF THE INVENTION

The present invention provides methods for relieving a symptom of inflammation in a subject, such as but not limited to redness, swelling, edema, excess, warmth and pain, the methods comprising administering to a subject in need thereof an effective amount of a theaflavin composition and an effective amount of one or more therapies other than such a theaflavin composition. The present invention also provides methods for preventing, treating, managing and/or ameliorating of a condition associated with inflammation (e.g., an inflammatory disorder) or a symptom thereof, the methods comprising administering to a subject in need thereof an effective amount of a theaflavin composition and an effective amount of one or more therapies other than such a theaflavin composition. Administration of such therapies can, for example, be via one or more compositions, food additives, dietary supplements, nutraceutical compositions or food compositions of the invention. In certain embodiments, the condition associated with inflammation is an acute condition. In other embodiments, the condition associated with inflammation is a chronic condition. Examples of inflammatory disorders that can be prevented, treated, managed and/or ameliorated in accordance with the invention include, but are not limited to, asthma, encephilitis, inflammatory bowel disease, chronic obstructive pulmonary disease (COPD), allergic disorders, septic shock, fibrosis (including, e.g., pulmonary fibrosis), undifferentitated spondyloarthropathy, undifferentiated arthropathy, arthritis, juvenile arthritis, psoriatic arthritis, rheumatoid arthritis, osteoarthritis, psoriasis, inflammatory osteolysis, degenerative joint diseases and chronic inflammation resulting from, e.g., chronic viral or bacteria infections.

The invention also provides methods for preventing, treating, managing and/or ameliorating an adverse health condition associated with the activation of NF-κB, the nuclear translocation of NF-κB, the binding of NF-κB to DNA in cells, and/or COX-2 gene expression, the methods comprising administering to a subject in need thereof an effective amount of a theaflavin composition and an effective amount of one or more therapies other than such a theaflavin composition. Administration of such therapies can, for example, be via one or more compositions, food additives, dietary supplements, nutraceutical compositions or food compositions of the invention. Examples of adverse health condition associated with the activation of NF-κB, the nuclear translocation of NF-κB, the binding of NF-κB to DNA in cells, and/or COX-2 gene expression, include, but are not limited to, an inflammatory disorder, or any symptoms associated with such disorders.

The present invention provides glucosamine compositions comprising glucosamine, or a pharmaceutically acceptable salt, solvate or hydrate thereof, which can be utilized in combination with a theaflavin composition of the invention to prevent, treat, manage and/or ameliorate a condition associated with inflammation (e.g., an inflammatory disorder). Non-limiting examples of salt forms of glucosamine include glucosamine sulfate, glucosamine hydrochloride, and n-acetyl glucosamine. In one embodiment, a glucosamine composition is or is from a natural source of glucosamine. In another, alternative embodiment, a glucosamine composition is not or is not from a natural source of glucosamine.

The therapies of the invention (L e., a theaflavin composition and at least one other therapy) can be administered to a subject in the same composition. Alternatively, the therapies can be administered concurrently to a subject in separate compositions. The therapies may be administered to a subject by the same or different routes of administration.

The present invention provides nutraceutical compositions comprising a theaflavin composition of the invention, and one or more prophylactic or therapeutic agents in addition to the theaflavin composition. In one embodiment, the nutraceutical compositions of the invention are prepared from natural sources.

The present invention also provides dietary supplements comprising a theaflavin composition of the invention, and one or more prophylactic or therapeutic agents in addition to the theaflavin composition. In one embodiment, the dietary supplements of the invention are prepared from natural sources.

The present invention also provides food additives comprising a theaflavin composition of the invention, and one or more prophylactic or therapeutic agents in addition to the theaflavin composition. In one embodiment, the food additives of the invention are prepared from natural sources.

The present invention also provides food compositions comprising a theaflavin composition of the invention, and one or more prophylactic or therapeutic agents in addition to the theaflavin composition. In one embodiment, the food compositions of the invention are prepared from natural sources.

The present invention also provides pharmaceutical compositions comprising a theaflavin composition of the invention, and one or more prophylactic or therapeutic agents in addition to the theaflavin composition. In one embodiment, such prophylactic or therapeutic agents are known to be useful, or have been or are currently being used to the prevention, treatment, management and/or amelioration of a condition associated with inflammation (e.g., an inflammatory disorder), or one or more symptoms thereof.

Theaflavin Compositions

The present invention provides theaflavin compositions comprising one or more of the theaflavins of the invention. The “theaflavins of the invention” include theaflavin (TF), theaflavin-3-gallate (TF-3-G), theaflavin-3′-gallate (TF-3′-G) and/or theaflavin-3,3′-digallate (TF-3,3′-diG), and pharmaceutically acceptable salts, solvates or hydrates thereof. In one embodiment, a theaflavin composition comprises one, two, three or all of the following theaflavins: TF, TF-3-G, TF-3′-G and/or TF-3,3′-diG, or pharmaceutically acceptable salts, solvates or hydrates thereof. In another embodiment, a theaflavin composition comprises any combination of the following theaflavins: TF, TF-3-G, TF-3′-G and/or TF-3,3′-diG, or pharmaceutically acceptable salts, solvates or hydrates thereof. In another embodiment, a theaflavin composition comprises TF, TF-3-G, TF-3′-G and TF-3,3′-diG, or pharmaceutically acceptable salts, solvates or hydrates thereof. In another embodiment, a theaflavin composition consists of or consists essentially of one two three or all of the following theaflavins: TF, TF-3-G, and/or TF-3,3′-diG, or a pharmaceutically acceptable salt, solvate or hydrate thereof; and a pharmaceutically acceptable carrier, vehicle or excipient. In another embodiment, a theaflavin composition is or is from a natural source of theaflavins (e.g., TF, TF-3-G, TF-3′-G and TF-3,3′-diG). In another, alternative embodiment, a theaflavin composition is not or is not from a natural source of theaflavins (e.g., TF, TF-3-G, TF-3′-G and TF-3,3′-diG). In another embodiment, a theaflavin composition is a black tea extract. In accordance with this embodiment, the black tea extract, in certain embodiments, is from an extract from Camellia sinensis, preferably the leaf of Camellia sinensis. In another, alternative embodiment, a theaflavin composition is not a black tea extract.

In one aspect, a theaflavin composition of the invention comprises: (i) a concentration of a theaflavin(s) (e.g., TF, TF-3-G, TF-3′-G and/or TF-3,3′-diG) that is different from that in a natural source of theaflavins; and/or (ii) the ratio of the concentration of one of the theaflavins (e.g., TF, TF-3-G, TF-3′-G and/or TF-3,3′-diG) in the therapy to that of another theaflavin in the therapy is different from that in a natural source of the theaflavins. Such a theaflavin composition of the invention can be prepared, for example, by processing a natural source of theaflavins (e.g., black tea extract) such that at least one particular theaflavin (e.g., TF, TF-3-G, TF-3′-G and/or TF-3,3′-diG) has been selectively removed, retained, or enriched. Alternatively, one or more purified theaflavins (e.g., TF, TF-3-G, TF-3′-G and/or TF-3,3′-diG) can be used to make such a composition. Such a composition can also be prepared, for example, by adding an amount of at least one theaflavin (e.g., TF, TF-3-G, TF-3′-G and/or TF-3,3′-diG) to a natural source or processed form of a natural source of the theaflavins.

In various embodiments, a theaflavin composition of the invention comprises a mixture of TF, TF-3-G, TF-3′-G and/or TF-3,3′-diG, wherein the concentration of one or more of the theaflavins is increased or decreased relative to that in a natural source of the theaflavins. In other embodiments, the invention provides a theaflavin composition comprising a mixture of TF, TF-3-G, TF-3′-G and/or TF-3,3′-diG, or pharmaceutically acceptable salts, solvates or hydrates thereof, wherein the percentages (by dry weight) of one or more theaflavins relative to the total content of theaflavins in the therapy is different from that in a natural source of the theaflavins. In yet other embodiments, a theaflavin composition of the invention comprises a mixture of TF, TF-3-G, TF-3′-G and/or TF-3,3′-diG, or pharmaceutically acceptable salts, solvates or hydrates thereof, wherein the ratio of certain theaflavins in the therapy is different from that in a natural source of theaflavins.

In certain embodiments, a theaflavin composition of the invention comprises approximately 2% to 95%, approximately 10% to 95%, approximately 25% to 95%, approximately 40% to 95%, approximately 60% to 95% by weight of one, two, three, all or any combination of the following theaflavins, or pharmaceutically acceptable salts, solvates or hydrates thereof: TF, TF-3-G, TF-3′-G and/or TF-3,3′-diGP. In other embodiments, a theaflavin composition of the invention comprises approximately 2%, approximately 5%, approximately 10%, approximately 15%, approximately 25%, approximately 28%, approximately 30%, approximately 35%, approximately 40%, approximately 45%, approximately 50%, approximately 60%, approximately 65%, approximately 75%, approximately 80%, approximately 85%, approximately 90% or approximately 95% by weight of one, two, three, all or any combination of the following theaflavins, or pharmaceutically acceptable salts, solvates or hydrates thereof: TF, TF-3-G, TF-3′-G and/or TF-3,3′-diGP.

In certain embodiments, a theaflavin composition comprises a mixture of TF, TF-3-G, TF-3′-G and TF-3,3′-diG, wherein TF is approximately 20%, approximately 25%, approximately 30%, approximately 35% or approximately 40% of the total content of theaflavins, TF-3-G is approximately 10%, approximately 15%, approximately 20% or approximately 25% of the total content of theaflavins, TF-3′-G is approximately 10%, approximately 15%, approximately 20% or approximately 25% of the total content of theaflavins, and TF-3,3′-diG is approximately 20%, approximately 25%, approximately 30%, approximately 35% or approximately 40% of the total content of theaflavins. In other embodiments, a theaflavin composition comprises a mixture of TF, TF-3-G, TF-3′-G and TF-3,3′-diG, wherein TF is approximately 20% to approximately 40%, approximately 25% to 45%, or approximately 30% to approximately 45% of the total content of theaflavins, TF-3-G is approximately 10% to approximately 30%, approximately 15% to approximately 30%, or approximately 20% to approximately 30% of the total content of theaflavins, TF-3′-G is approximately 10% to approximately 30%, approximately 15% to approximately 30%, or approximately 20% to approximately 30% of the total content of theaflavins, and TF-3,3′-diG is approximately 20% to approximately 40%, approximately 25% to 45%, or approximately 30% to approximately 45% of the total content of theaflavins. In certain aspects, in accordance with these embodiments, the total content of theaflavins is approximately 2%, approximately 5%, approximately 10%, approximately 15%, approximately 25%, approximately 28%, approximately 30%, approximately 35%, approximately 40%, approximately 45%, approximately 50%, approximately 60%, approximately 65%, approximately 75%, approximately 80%, approximately 85%, approximately 90% or approximately 95%.

In certain embodiments, a theaflavin composition comprises a theaflavin component and a non-theaflavin component. In certain embodiments, the non-theaflavin component comprises a pharmaceutically acceptable carrier, vehicle or excipient. In certain embodiments, the non-theaflavin component comprises an active ingredient such as a compound with anti-inflammatory activity. In other embodiments, the non-theaflavin component does not comprise a compound with anti-inflammatory activity as detectable in an in vitro assay and/or an animal model for inflammation. In a specific embodiment, the non-theaflavin component does not have anti-inflammatory activity as measured by the inhibition of carrageenan-induced paw edema in the rat, as described infra and known in the art. In certain embodiments, the theaflavin component consists of or consists essentially of one, two, three or all of the following theaflavins: TF, TF-3-G, TF-3′-G and/or TF-3,3′-diG, or pharmaceutically acceptable salts, solvates or hydrates thereof.

In certain embodiments, a theaflavin composition consists of or consists essentially of a theaflavin(s) described herein, and a pharmaceutically acceptable carrier, vehicle or excipient. In other embodiments, a theaflavin composition comprises a theaflavin(s) described herein, as the active ingredient, and a pharmaceutically acceptable carrier, vehicle or excipient. In accordance with these embodiments, the theaflavin composition does not comprise active ingredients other than the theaflavins described herein.

In certain embodiments, a theaflavin composition comprises approximately 2% to 95%, approximately 10% to 95%, approximately 25% to 95%, approximately 40% to 95%, approximately 60% to 95% by weight of one, two, three, all or any combination of the following theaflavins, or pharmaceutically acceptable salts, solvates or hydrates thereof: TF, TF-3-G, TF-3′-G and/or TF-3,3′-diGP; and approximately 2% to 65%, approximately 10% to 65%, approximately 25% to 95% or approximately 40% to 95% by weight of catechins. In certain aspects, in accordance with these embodiments, the theaflavin composition further comprises less than 2.5% (in some embodiments, less than 1.5% or less than 1%) by weight of caffeine.

In certain embodiments, a theaflavin composition does not comprise a rosemary extract, a Mexican Bamboo extract, a Huzhang extract, resveratrol, a hydroxylated resveratrol analog, a methoxylated resveratrol analog, a green tea extract, a catechin, an orange peel extract or a polymethoxylated flavone found in an orange peel extract. In yet other embodiments, a theaflavin composition does not comprise glucosamine.

The theaflavin composition that is administered to a subject to prevent, treat, manage and/or ameliorate a condition associated with inflammation can be in the form of or incorporated into a food additive, dietary supplement, nutraceutical composition or food composition.

Methods for Producing Theaflavin Therapies

In one embodiment, theaflavin (TF), theaflavin-3-gallate (TF-3-G), theaflavin 3′-gallate (TF-3′-G) and/or theaflavin-3,3′-digallate (TF-3,3′-diG) can be extracted from a natural source. Typically, the natural source or preferred portions thereof, such as the leaves or floral parts of a plant, in natural or dried form, may be used directly, or pulverized, ground or comminuted to a fine powder in order to maximize surface contact with the solvent. The methods of the present invention can be employed on any known plant matter or “biomass” containing an appreciable amount of theaflavin (TF), theaflavin-3-gallate (TF-3-G), theaflavin 3′-gallate (TF-3′-G) and/or theaflavin-3,3′-digallate (TF-3,3′-diG).

According to the methods of the present invention, the biomass is contacted with a water miscible solvent sufficient to put theaflavins of the invention (i.e., theaflavin (TF), theaflavin-3-gallate (TF-3-G), theaflavin 3′-gallate (TF-3′-G) and/or theaflavin-3,3′-digallate (TF-3,3′-diG)) in the biomass into solution. The solvent solubilizes the theaflavins of the invention and preferably it is water miscible. Solvent extraction can be performed at room temperature or at elevated temperatures, usually at from about 3° C. to about 70° C., with or without ultrasonication. Suitable solvents include, but are not limited to, acetone and ethanol. Methanol or isopropanol may also be used, but are generally undesirable in preparing compositions for human food applications. Other solvent that can be used include but is not limited to carbon tetrachloride, cyclohexane, toluene, dichloromethane, chloroform, diethyl ether, diisopropyl ether, ethyl acetate, butanol, n-propanol, polyethylene glycol, propylene glycol, pyridine, and the like. A mixture of two or more solvents can be used. The biomass:solvent ratio should be at a minimum 2 L of extracting solvent to 1 kg of biomass. For example, the solvent(s) was added to the biomass at room temperature and ultrasonicated for one hour, and then shaken for 30 minutes to carry out the extraction. The degree of extraction of the compounds is generally greater than 80%, while the theaflavins purity in the solids of the extract is between 1% and 10%. The solvent can be removed by distillation under reduced pressure.

In various embodiments, theaflavins of the invention and the insoluble impurities can then be separated by any conventional means, such as filtration or centrifugation. The present invention provides additional purifications employing selective adsorption to resins and elution.

In a specific embodiment, theaflavins of the invention can be selectively removed, enriched or retained by applying supercritical fluid extraction technique to a natural source. This technique, which generally utilizes carbon dioxide, is known in the art, especially for preparing food and medicinal substances for human consumption. See, for example, Hamburger et al., Phytochemical Analysis (2004), 15(1), 46-54; Simandi et al., Recents Progres en Genie des Procedes (1999) 13(71), 157-164, the disclosures of which are incorporated herein by reference in their entirety.

In a specific embodiment, theaflavin (TF), theaflavin-3-gallate (TF-3-G), theaflavin 3′-gallate (TF-3′-G) and/or theaflavin-3,3′-digallate (TF-3,3′-diG) are extracted from black tea. In accordance with this embodiment, such theaflavins can be extracted from black tea utilizing the techniques described in U.S. Publication No. 2002/014672 A1, published Oct. 10, 2002, which is incorporated herein in its entirety. Briefly, black tea powder (100 g) is soaked in hot water (1000 ml) for 10 minutes. After filtration, the filtrates are extracted with 300 ml of chloroform three times for decaffeination. The aqueous phase is collected and extracted twice with 300 ml of ethyl acetate. The combined ethyl acetate phases is washed with a 2.5% sodium bicarbonate solution (300 ml) followed by distilled water (500 ml ). The crude theaflavins (1.5 to 3%) are obtained after evaporating ethyl acetate to dryness in a vacuum rotary evaporator.

In another embodiment, the theaflavins of the invention can be synthesized chemically. In a specific embodiment, the theaflavins of the invention are synthesized utilizing the techniques described in U.S. Publication No. 2005/0049284 A1, published Mar. 3, 2005, which is incorporated herein in its entirety.

In certain embodiments, TF is synthesized as follows: epicaechin (EC) (1.0 g) and epigallocatechin (EGC) (1.0 g) are dissolved in a mixture of acetone-pH 5.0 phosphate-citrate buffer (1:10 v/v, 50 mL), which contains 4 mg horseradish peroxidase. Four 2.0-ml aliquots of 3.13% H₂O₂ are added during a period of 45 minutes while stirring. The resulting reaction mixture is extracted with ethyl acetate (3×50 mL). After concentration, the residue is applied to a Sephadex LH 20 column and eluted with acetone-water (2:3 v/v) to obtain TF (in certain embodiments, 250 mg of TF).

In certain embodiments, TF-3-G is synthesized following the procedure described in paragraph 79 for TF synthesis using epicatechin (EC) (1.0 g) and epigallocatechin gallate (EGCG) (1.0 g). In certain aspects, in accordance with these embodiments, the procedure results in the synthesis of 220 mg of TF-3-G.

In certain embodiments, TF-3′-G is synthesized following the procedure described in paragraph 79 for TF synthesis using epicatechin gallate (ECG) (1.0 g) and epigallocatechin (EGC) (1.0 g). In certain aspects, in accordance with these embodiments, the procedure results in the synthesis of 110 mg of TF-3′-G.

In certain embodiments, TF-3,3′-G is synthesized following the procedure described in paragraph 79 for TF synthesis using epicatechin gallate (ECG) (1.0 g) and epigallocatechin gallate (EGCG) (1.0 g). In certain aspects, in accordance with these embodiments, the procedure results in the synthesis of 100 mg of TF-3,3′-G.

Once synthesized, a theaflavin of the invention can be isolated from chemical precursors or other chemicals using standard purification techniques such as, for example, chromatography (e.g., flash column chromatography and HPLC), recrystallization, and differential solubility.

Therapies Useful In Combination With a Theaflavin composition

The present invention provides methods for preventing, managing, treating, and/or ameliorating a condition associated with inflammation (e.g., inflammatory disorders), the methods comprising administering to a subject in need thereof a theaflavin composition of the invention and one or more therapies (e.g., one or more prophylactic or therapeutic agents) other than such a theaflavin composition. In a specific embodiment, the present invention provides methods for preventing, managing, treating and/or ameliorating a condition associated with inflammation, the methods comprising administering to a subject in need thereof one two three or all of the following theaflavins: TF, TF-3-G, TF-3′-G and/or TF-3,3′-diG, or a pharmaceutically acceptable salt, solvate or hydrate thereof; and one or more other therapies other than such theaflavins. Any therapy which contributes to the prevention, management, treatment, and/or amelioration of a condition associated with inflammation (e.g., an inflammatory disorder) or one or more symptoms thereof can be used in combination with a theaflavin composition of the invention in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996; Physician's Desk Reference for Herbal Medicine (2003); and Physician's Desk Reference (59^{th} edition, 2005) (each of which are incorporated herein by reference in their entirety) for information regarding therapies which have been or are currently being used for preventing, treating, managing and/or ameliorating conditions associated with inflammation (e.g., inflammatory disorders) or one or more symptoms thereof. Non-limiting examples of therapies that can be utilized in combination with a theaflavin composition to prevent, treat, manage and/or ameliorate a condition associated with inflammation (e.g., inflammatory disorders) or one or more symptoms thereof include glucosamine, methylsulfonylmethane, Bowellia extract, bromelain, tumeric extract, Feverfew, hops, phellodendron, devil's claw extract, gamma-linolenic acid, cat's claw, cis-9-cetylmyristoleate, chondroitin, collagen, fish oil, omega-3 fatty acids, ginger, ginkgo biloba, ginseng, gotu kola, grapeseed, gugulipid, melatonin, Noni, New Zealand green-lipped mussel, S-adenosyl-L-methionine, white willow bark, stinging nettle, deer antler velvet, Vitamin B3, Vitamin C, Vitamin E, boron, superoxide dismutase, back cohosh, cayenne, meadowsweet, alfalfa, yucca apple cider vinegar, cherry juice, hylaronic acid, celadrin, methotrexate, TNF-α antagonists, non-steroidal anti-inflammatory drugs (NSAIDs), steroidal anti-inflammatory drugs, opioid medications, adrenergic stimulants, resorcinols, saligenins, anticholinergics, beta2-agonists, leukotriene modifiers, mast cell stabilizers, methylxanthines, mucolytic agents, antihistamines, decongestants, anti-viral agents, and antibiotics. See Sections 4.2.1-4.2.6 and Section 4.3, infra, for additional information regarding therapies to be utilized in combination with a theaflavin composition.

In a specific embodiment, a therapy that reduces or slows down cartilage degradation is utilized in combination with a theaflavin composition of the invention to prevent, treat, manage and/or ameliorate a condition associated with inflammation or a symptom thereof. In another embodiment, a therapy that facilitates the rebuilding of cartilage is utilized in combination with a theaflavin composition of the invention to prevent, treat, manage and/or ameliorate a condition associated with inflammation or a symptom thereof.

The present invention also provides compositions comprising a theaflavin composition and one or more other prophylactic or therapeutic agents, and methods of preventing, managing, treating and/or ameliorating a condition associated with inflammation (e.g., an inflammatory disorder) utilizing such compositions. In a specific embodiment, the invention provides compositions comprising one, two, three or all of the following theaflavins: TF, TF-3-G, TF-3′-G, and/or TF-3,3′-diG, or a pharmaceutically acceptable salt, solvate or hydrate thereof; and one or more other prophylactic or therapeutic agents. Examples of the types of therapeutic or prophylactic agents that can be utilized in such compositions include, but are not limited to, plant extracts, small molecules, synthetic drugs, peptides, polypeptides, proteins, nucleic acids (e.g., DNA and RNA nucleotides including, but not limited to, antisense nucleotide sequences, RNAi, triple helices and nucleotide sequences encoding biologically active proteins, polypeptides or peptides), antibodies, synthetic or natural inorganic molecules, mimetic agents, and synthetic or natural organic molecules. Non-limiting examples of prophylactic and therapeutic agents that can be included in a composition comprising a theaflavin composition are provided in Sections 4.2.1-4.2.6, infra. In a specific embodiment, a composition comprises a theaflavin composition, and one or more natural products, phytochemicals and/or botanical extracts, other than such a theaflavin composition. In a preferred embodiment, a composition comprises a theaflavin composition, a glucosamine composition and optionally, a therapy other than a theaflavin composition and glucosamine composition. Depending on the manner of use, the compositions of the invention can be, but not limited to, a dietary supplement, a food additive, a pharmaceutical composition, or a cosmetic composition.

4.2.1 Immunodulatory Agents

Any immunomodulatory agent well-known to one of skill in the art may be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not a theaflavin of the invention. In certain embodiments, an immunomodulatory agent is not an anti-inflammatory agent. In other embodiments, an immunomodulatory agent is not a TNF-α antagonist.

In certain embodiments, an immunomodulatory agent is a chemotherapeutic agent. In other embodiments, an immunomodulatory agent is not a chemotherapeutic agent.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules, triple helices and nucleic acid molecules encoding immunomodulatory gene products), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunomide), T cell receptor modulators, and cytokine receptor modulators.

As used herein, the term “T cell receptor modulator” refers to an agent which modulates the phosphorylation of a T cell receptor, the activation of a signal transduction pathway associated with a T cell receptor, the differentiation of a T cell and/or the expression of a particular protein such as a cytokine. Such an agent may directly or indirectly modulate the phosphorylation of a T cell receptor, the activation of a signal transduction pathway associated with a T cell receptor, and/or the expression of a particular protein such as a cytokine. Thus, examples of T cell receptor modulators include, but are not limited to, peptides, polypeptides, proteins, fusion proteins and antibodies which immunospecifically bind to a T cell receptor or a fragment thereof. Further, examples of T cell receptor modulators include, but are not limited to, proteins, peptides, polypeptides (e.g., soluble T cell receptors), fusion proteins and antibodies that immunospecifically binds to a ligand for a T cell receptor or a fragment thereof. Specific examples of T cell receptor modulators include, but are not limited to, anti-T cell receptor antibodies (e.g., anti-CD2 antibodies, anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD11 a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

As used herein, the term “cytokine receptor modulator” refers to an agent which modulates the phosphorylation of a cytokine receptor, the activation of a signal transduction pathway associated with a cytokine receptor, the proliferation and/or differentiation of a cell expressing a cytokine receptor, and/or the expression of a particular protein such as a cytokine. Such an agent may directly or indirectly modulate the phosphorylation of a cytokine receptor, the activation of a signal transduction pathway associated with a cytokine receptor, the proliferation and/or differentiation of a cell expressing a cytokine receptor, and/or the expression of a particular protein such as a cytokine. Thus, examples of cytokine receptor modulators include, but are not limited to, cytokines, fragments of cytokines, fusion proteins and antibodies that immunospecifically binds to a cytokine receptor or a fragment thereof. Further, examples of cytokine receptor modulators include, but are not limited to, peptides, polypeptides (e.g., soluble cytokine receptors), fusion proteins and antibodies that immunospecifically binds to a cytokine or a fragment thereof. Specific examples of cytokine receptor modulators include, but are not limited to, soluble cytokine receptors (e.g., the extracellular domain of a TNF-a receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, IL-23 TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, anti-IL-12 receptor antibodies, anti-IL-15 receptor antibodies and anti-IL-23 receptor antibodies), anti-cytokine antibodies (e.g., anti-IFN α antibodies, anti-IFN-β antibodies, anti-IFN-γ antibodies, anti-TNF-α antibodies, anti-IL-β antibodies, anti-IL-2 antibodies, anti-IL-4 antibodies, anti-IL-6 antibodies, anti-IL-8 antibodies (e.g., ABX-IL-8 (Abgenix)), anti-IL-9 antibodies, anti-IL-10 antibodies, anti-IL-12 antibodies and anti-IL-23 antibodies). In a specific embodiment, a cytokine receptor modulator is IL-4, IL-10, or a fragment thereof. In another embodiment, a cytokine receptor modulator is an anti-IL-β antibody, anti-IL-6 antibody, anti-IL-12 receptor antibody, or anti-TNF-α antibody. In another embodiment, a cytokine receptor modulator is the extracellular domain of a TNF-α a receptor or a fragment thereof. In certain embodiments, a cytokine receptor modulator is not a TNF-α antagonist.

An immunomodulatory agent may be selected to interfere with the interactions between the T helper subsets (TH1 or TH2) and B cells to inhibit neutralizing antibody formation. An immunomodulatory agent may also be selected to inhibit the interaction between TH1 cells and cytoxic T cells (CTSs) to reduce the occurrence of CTL-mediated killing. Further, an immunomodulatory agent may be selected to alter (e.g., inhibit or suppress) the proliferation, differentiation, activity and/or function of the CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells.

In one embodiment of the invention, an immunomodulatory agent that reduces or depletes T cells, preferably memory T cells, is administered to a subject with a condition associated with inflammation (e.g., an inflammatory disorder) in accordance with the methods of the invention. See, e.g., U.S. Pat. No. 4,658,019. In another embodiment of the invention, an immunomodulatory agent that inactivates CD8+ T cells is administered to a subject with a proliferative disorder or an inflammatory disorder in accordance with the methods of the invention. In a specific embodiment, anti-CD8 antibodies are used to reduce or deplete CD8+ T cells.

Antibodies that interfere with or block the interactions necessary for the activation of B cells by TH (T helper) cells, and thus block the production of neutralizing antibodies, are useful as immunomodulatory agents in accordance the methods of the invention. For example, B cell activation by T cells requires certain interactions to occur (Ducie et al, Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

In another embodiment, an immunomodulatory agent which reduces or inhibits one or more biological activities (e.g., the differentiation, proliferation, and/or effector functions) of Th0, TH1, and /or TH2 subsets of CD4⁺ T helper cells is administered to a subject with a condition associated with inflammation (e.g., an inflammatory disorder) in accordance with the methods of the invention. One example of such an immunomodulatory agent is IL-4. IL-4 enhances antigen-specific activity of TH2 cells at the expense of the TH1 cell function (see, e.g., Yokota et al., 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-9, IL-10, IL-12, IL-13, IL-15, IL-18 and interferon (IFN)-γ.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

In accordance with the invention, one or more immunomodulatory agents are administered to a subject with a condition associated with inflammation (e.g., an inflammatory disorder) prior to, subsequent to, or concomitantly with a theaflavin composition. Preferably, one or more immunomodulatory agents are administered to a subject with an inflammatory disorder in combination with a theaflavin composition to reduce or inhibit one or more aspects of the immune response. Any technique well-known to one skilled in the art can be used to measure one or more aspects of the immune response in a particular subject, and thereby determine when to administer an immunomodulatory agent to said subject. In a preferred embodiment, a mean absolute lymphocyte count of approximately 500 cells/mm³, preferably 600 cells/mm³, 650 cells/mm³, 700 cells/mm³, 750 cells/mm³, 800 cells/mm³, 900 cells/mm³, 1000 cells/mm³, 1100 cells/mm³, or 1200 cells/mm³ is maintained in a subject. In another preferred embodiment, a subject with a condition associated with inflammation (e.g., an inflammatory disorder) is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered to a subject with a condition associated with inflammation (e.g., an inflammatory disorder) in combination with a theaflavin composition so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response last for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the immune response enhances the prophylactic and/or therapeutic capabilities of a theaflavin composition.

Nucleic acid molecules encoding proteins, polypeptides, or peptides with immunomodulatory activity or proteins, polypeptides, or peptides with immunomodulatory activity can be administered to a subject with a condition associated with inflammation (e.g., an inflammatory disorder) in accordance with the methods of the invention. Further, nucleic acid molecules encoding derivatives, analogs, or fragments of proteins, polypeptides, or peptides with immunomodulatory activity, or derivatives, analogs, or fragments of proteins, polypeptides, or peptides with immunomodulatory activity can be administered to a subject with a condition associated with inflammation (e.g., an inflammatory disorder) in accordance with the methods of the invention. Preferably, such derivatives, analogs, and fragments retain the immunomodulatory activity of the full-length, wild-type protein, polypeptide, or peptide.

Proteins, polypeptides, or peptides that can be used as immunomodulatory agents can be produced by any technique well-known in the art or described herein. Proteins, polypeptides or peptides with immunomodulatory activity can be engineered so as to increase the in vivo half-life of such proteins, polypeptides, or peptides utilizing techniques well-known in the art or described herein. Preferably, agents that are commercially available and known to function as immunomoulatory agents are used in the compositions and methods of the invention. The immunomodulatory activity of an agent can be determined in vitro and/or in vivo by any technique well-known to one skilled in the art, including, e.g., by CTL assays (⁵¹Cr release assays), proliferation assays (³H-thymidine incorporation or trypan blue cell counts), northern blot assays, and immunoassays (e.g., ELISAs and western blot expression) for the expression of particular gene products (e.g., RNA or proteins) such as co-stimulatory molecules and cytokines.

Immunomodulatory agents and their dosages, routes of administration and recommended usage are known in the art and have been described in such literature as the Physician's Desk Reference (59th ed., 2005).

TNF-α Antagonists

Any TNF-a antagonist well-known to one of skill in the art can be used in the compositions and methods of the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that block, reduce, inhibit or neutralize a function, an activity and/or the expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS). In accordance with this embodiment, the activity of TNF-α can be determined by measuring the expression of gene that is regulated by TNF-α using assays well-known in the art (e.g., RT-PCR, Northern blots, and immunoassays such as ELISAs and Western blots). Further, in accordance with this embodiment, the expression of TNF-α can be determined by using assays well-known in the art (e.g., RT-PCR, Northern blots, and immunoassays such as ELISAs and Western blots).

Examples of antibodies that immunospecifically bind to TNF-α include, but are not limited to, infliximab (REMICADE®; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of the antibodies that immunospecifically bind to TNF-a disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al., 1990, Proc. Natl. Acad. Sci. USA 87:5188-5192).

In one embodiment, a TNF-α antagonist used in the compositions and methods of the invention is a soluble TNF-α receptor. In a specific embodiment, a TNF-α antagonist used in the compositions and methods of the invention is etanercept (ENBREL™; Immunex) or a fragment, derivative or analog thereof. In another embodiment, a TNF-α antagonist used in the compositions and methods of the invention is an antibody that immunospecifically binds to TNF-α. In a specific embodiment, a TNF-α antagonist used in the compositions and methods of the invention is infliximab (REMICADE®; Centacor) a derivative, analog or antigen-binding fragment thereof.

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Nucleic acid molecules encoding proteins, polypeptides, or peptides with TNF-α antagonist activity, or proteins, polypeptides, or peptides with TNF-α antagonist activity can be administered to a subject with a condition associated with inflammation (e.g., an inflammatory disorder) in accordance with the methods of the invention. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered to a subject with a condition associated with inflammation (e.g., an inflammatory disorder) in accordance with the methods of the invention. Preferably, such derivatives, analogs, variants and fragments retain the TNF-α antagonist activity of the full-length, wild-type protein, polypeptide, or peptide.

Proteins, polypeptides, or peptides that can be used as TNF-α antagonists can be produced by any technique well-known in the art or described herein. Proteins, polypeptides or peptides with TNF-α antagonist activity can be engineered so as to increase the in vivo half-life of such proteins, polypeptides, or peptides utilizing techniques well-known in the art or described herein. Preferably, agents that are commercially available and known to function as TNF-α antagonists are used in the compositions and methods of the invention. The TNF-α antagonist activity of an agent can be determined in vitro and/or in vivo by any technique well-known to one skilled in the art.

TNF-α antagonists and their dosages, routes of administration and recommended usage are known in the art and have been described in such literature as the Physician's Desk Reference (59th ed., 2005).

Anti-Inflammatory Agents

Anti-inflammatory agents have exhibited success in treatment of conditions associated with inflammation (e.g., inflammatory disorders) and are now a common and a standard treatment for such conditions. Any anti-inflammatory therapy (e.g., an anti-inflammatory agent) well-known to one of skill in the art can be used in the compositions and methods of the invention. Non-limiting examples of anti-inflammatory agents include non-steroidal anti-inflammatory drugs (NSAIDs), steroidal anti-inflammatory drugs, beta-agonists, anticholingeric agents, antihistamines (e.g., ethanolamines, ethylenediamines, piperidines, alkylamaines, piperazines, and phenothiazine), and methyl xanthines. Examples of NSAIDs include, but are not limited to, aspirin, ibuprofen, salicylates, acetominophen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Anti-inflammatory agents and their dosages, routes of administration and recommended usage are known in the art and have been described in such literature as the Physician's Desk Reference (59th ed., 2005).

Antibiotics

Antibiotics well known to one of skill in the art can be used in the compositions and methods of the invention. Non-limiting examples of antibiotics include penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, spectinomycin, trimethoprim, norfloxacin, rifampin, polymyxin, amphotericin B, nystatin, ketocanazole, isoniazid, metronidazole, and pentamidine.

Antibiotics and their dosages, routes of administration and recommended usage are known in the art and have been described in such literature as the Physician's Desk Reference (59th the proliferation and/or differentiation of a cell expressing a cytokine receptored., 2005).

Antiviral Agents

Any anti-viral agent well-known to one of skill in the art can be used in the compositions and the methods of the invention. Non-limiting examples of anti-viral agents include proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons and other interferons, and AZT.

Antiviral agents and their dosages, routes of administration and recommended usage are known in the art and have been described in such literature as the Physician's Desk Reference (59th ed., 2005).

Natural Products and their Derivatives

In the present invention, the theaflavins compositions can be administered either alone or in combination with natural products and their derivatives. Further, the invention provides compositions comprising a theaflavin composition and natural products and their derivatives. In a specific embodiment, the invention provides compositions comprising one two three or all of the following theaflavins: TF, TF-3-G, TF-3′-G and TF-3,3′-diG, or a pharmaceutically acceptable salt, solvate or hydrate thereof; and a natural product or a derivative thereof. In a particular embodiment, natural products and derivatives that have immunomodulatory activity, anti-TNFα activity, anti-inflammatory activity, anti-infective activity and/or antiviral activity are used in combination with the theaflavin compositions of the invention. Many such natural products are present in mixtures and are not fully characterized by their chemical structures and/or properties, e.g., botanical extracts, juices, powders, leaves, roots, fruits and seeds. Some have been functionally characterized with their active ingredients partially identified. Non-limiting examples of such extracts, juices, and powders include, but are not limited to, licorice root extracts, Inula extracts, Boswellia extracts (e.g., extracts from the Boswellia serrata tree), tumeric extracts, feverfew (e.g., fresh or dried leaf extracts from the feverfew plant (Tanacetum parthenium), green tea extract (extracted from leaf buds and young leaves of a tea plant), devil's claw extracts (e.g., extracts from harpagophytum procumbens), tinging nettle extract (e.g., extract of leaves and/or roots of Urtica dioica), black cohosh (found in root of Cimicifuga racemosa), meadowsweet extract, Noni (juice of Morinda citrifolia), white willow bark (bark of Salix alba), gotu kola (herb from Centella asiatica), alfalfa, cherry juice, apple cider vinegar, phellodendron, cayenne (capsicum), extract of yucca (e.g., a saponin extract of yucca), grapeseed, ginger (Zingiber officinale Roscoe), Rabdosia (Rabdosia rubescens Hara), cranberry (Vaccinium macrocarpon L. and other species of cranberry), lesser galangal (Alpinia officinarum Hance), wild garlic (Allium ursinum L.), licorice (Glycyrrhiza glabra L.), soy and cat's claw (e.g., extracts from Uncaria tomentosa). In certain embodiments, the extract is not a rosemary extract, a Mexican Bamboo extract, a Huzhang extract, a green tea extract or an orange peel extract.

Purified compounds found in juices, fruit, bark, extracts, leaves, seeds, roots and powders from various vegetation and derivatives thereof can be used in accordance with the invention. Non-limiting examples of such compounds include methylsulfonylmetane (found in fruits, vegetables, grains and animals), bromelain (an enzyme found in pineapple juice), hops (found in the hops plant natibe to Europe, Asia and North America), gamma-linolenic acid (found in evening primrose oil, black current oil and borage oil), ginger, ginkgo biloba (extracted from leaf of the gingko tree), ginseng (extracted from root of ginseng plant), gugulipid (gum resin from the guggul tree), salicin (found in flower buds of meadowsweet), and celadrin. In certain embodiments, the compound is not a polymethoxylated flavone, a polymethoxylated flavone found in an orange peel extract, resveratrol, a hydroxylated resveratrol analog, a methoxylated resveratrol analog and/or a catechin.

Animal extracts (e.g., fish, human, and non-human mammalian extracts), compounds purified from such extracts, and derivatives of such compounds can be used in accordance with the invention. Non-limiting examples of such extracts and compounds include glucosamine, cis-9-cetylmyristoleate, chondroitin, collagen, gelatin, fish oil, omega-3 fatty acids, melatonin, New Zealand green-lipped mussel (a lipid extract or freeze-dried concentrate of New Zealand shellfish), S-adenosyl-L-methionine, shark cartilage, deer antler velvet (soft cartinaginous tissue from red deer or elk), superoxide dismutase, creatine and hylaronic acid.

Vitamins, minerals and amino acids can be used in accordance with the invention. Non-limiting examples of vitamins include Vitamin B3, Vitamin C and Vitamin E. Non-limiting examples of minerals include boron. Non-limiting examples of amino acids include L-arginine.

Non-limiting examples of phytochemicals or plant extracts that can be used in combination with the compounds and compositions of the invention are disclosed in U.S. Pat. Nos. 6,498,195, 6,627,623, and 6,790,869, and International patent publications Nos. WO 01/21137 and WO 02/39956, which are incorporated herein by reference in their entirety.

Natural products and their dosages, routes of administration and recommended usage are known in the art and have been described in such literature as the Physician's Desk Reference for Herbal Medicines (2003).

Glucosamine Compositions

The present invention provides glucosamine compositions comprising glucosamine, or a pharmaceutically acceptable salt, solvate or hydrate thereof, which can be utilized in combination with a theaflavin composition of the invention to prevent, treat, manage and/or ameliorate a condition associated with inflammation (e.g., an inflammatory disorder). In a preferred embodiment, the present invention provides methods for preventing, treating, managing and/or ameliorating a condition associated with joint inflammation (e.g., osteoarthritis, arthritis and other degenerative joint diseases), the methods comprising administering to a subject in need thereof an effective amount of a glucosamine composition comprising glucosamine, or a pharmaceutically acceptable salt, solvate or hydrate thereof, an effective amount of a theaflavin composition, and optionally one or more other therapies.

In a specific embodiment, a glucosamine composition comprises a salt form of glucosamine. Non-limiting examples of such salt forms of glucosamine include glucosamine sulfate, glucosamine hydrochloride, and n-acetyl glucosamine. In one embodiment, a glucosamine composition is or is from a natural source of glucosamine. In another, alternative embodiment, a glucosamine composition is not or is not from a natural source of glucosamine.

In certain embodiments, a glucosamine composition comprises glucosamine, or a pharmaceutically acceptable salt, solvate or hydrate thereof, and chondroitin, or a pharmaceutically acceptable salt, solvate or hydrate thereof (e.g., chondroitin sulfate). In other embodiments, a glucosamine composition comprises glucosamine, or a pharmaceutically acceptable salt, solvate or hydrate thereof, and methylsulfonylmethane. In yet other embodiments, a glucosamine composition comprises glucosamine, or a pharmaceutically acceptable salt, solvate or hydrate thereof, chondroitin, or a pharmaceutically acceptable salt, solvate or hydrate thereof, and methylsulfonylmethane.

In certain embodiments, a glucosamine composition comprises a glucosamine component and a non-glucosamine component. In certain embodiments, the non-glucosamine component comprises a pharmaceutically acceptable carrier, vehicle or excipient. In certain embodiments, the non-glucosamine component comprises an active ingredient such as a compound with anti-inflammatory activity. In other embodiments, the non-glucosamine component does not comprise a compound with anti-inflammatory activity, as detectable in an in vitro and/or an animal model for inflammation. In a specific embodiment, the non-glucosamine component does not have anti-inflammatory activity as measured by the inhibition of carrageenan-induced paw edema in the rat, as described infra and known in the art. In certain embodiments, the glucosamine component consists of or consists essentially of glucosamine or a pharmaceutically acceptable salt, solvate or hydrate thereof.

In certain embodiments, a glucosamine composition consists of or consists essentially of a glucosamine, or a pharmaceutically acceptable salt, solvate or hydrate thereof, and a pharmaceutically acceptable carrier, vehicle or excipient. In other embodiments, a glucosamine composition comprises glucosamine, as the active ingredient, and a pharmaceutically acceptable carrier, vehicle or excipient. In accordance with these embodiments, the glucosamine composition does not comprise active ingredients other than glucosamine.

In certain embodiments, a glucosamine composition of the invention comprises approximately 10% to 98%, approximately 20% to 98%, approximately 40% to 98%, approximately 50% to 98%, approximately 60% to 98% by weight of glucosamine, or a pharmaceutically acceptable salt, solvate or hydrate thereof. In other embodiments, a composition of the invention comprises a theaflavin composition and approximately 10%, approximately 15%, approximately 25%, approximately 30%, approximately 40%, approximately 50%, approximately 60%, approximately 75%, approximately 95% or approximately 98% by weight of glucosamine, or a pharmaceutically acceptable salt, solvate or hydrate thereof. In other embodiments, a composition of the invention comprises approximately 10%, approximately 15%, approximately 25%, approximately 30%, approximately 40%, approximately 50%, approximately 60%, approximately 75%, approximately 95% or approximately 98% by weight of glucosamine, or a pharmaceutically acceptable salt, solvate or hydrate thereof and one, two, three or all of the following theaflavins or a pharmaceutically acceptable salt, solvate or hydrate thereof: TF, TF-3-G, TF-3′-G and/or TF-3,3 -diG.

A glucosamine composition that is administered to a subject to prevent, treat, manage and/or ameliorate a condition associated with inflammation can be in the form of or incorporated into a food additive, dietary supplement, nutraceutical composition or food composition.

Nutraceutical Compositions

The present invention provides compositions for the treatment, prophylaxis, and amelioration of a condition associated with inflammation (e.g., an inflammatory disorder) in a subject. In one embodiment, a composition is a theaflavin composition, such as described in Section 4.1, supra. In another embodiment, a composition is a glucosamine composition such as described in Section 4.3, supra. In another embodiment, a composition comprises a theaflavin composition, and one or more prophylactic or therapeutic agents other than a theaflavin composition. In another embodiment, a composition comprises a theaflavin composition, a glucosamine composition, and optionally one or more other therapies other than a theaflavin composition and glucosamine composition. In another embodiment, a composition comprises: (a) glucosamine, or a pharmaceutically acceptable salt, solvate or hydrate thereof; (b) one, two or three or all of the following theaflavins or a pharmaceutically acceptable salt, solvate or hydrate thereof; theaflavin, theaflavin-3-gallate, theaflavin-3′-gallate, and theaflavin-3,3′-digallate; and (c) optionally one or more other therapies.

In one embodiment, a composition described herein is a nutraceutical composition. As used herein, the terms “nutraceutical” or “nutraceutical composition of the invention” are used interchangeably to refer to, without limitation, food compositions, food additives, food compositions in bulk, food additives in bulk, dietary supplements, medical foods, and foods for special dietary use, of the invention. In various embodiments, a nutraceutical composition of the invention typically comprises one or more consumable vehicles, carriers, excipients, or fillers. The term “consumable” means generally suitable for, or is approved by a regulatory agency of the Federal or a state government for, consumption by animals, and more particularly by humans.

As used herein, “food” means any substance, whether processed, semi-processed, or raw, which is intended for consumption by animals including humans, and includes but is not limited to drink, chewing gum and any substance which has been used in the manufacture, preparation of treatment of treatment of food, but does not include cosmetics, tobacco products or substances used only as pharmaceuticals. The term “food composition” refers to a food to which a composition described herein is added, or a food in which a composition described herein is made to be present at a greater level.

In one embodiment, the invention provides a food additive comprising a composition described herein. As used herein, the term “food additive” refers to any substance not normally consumed as a food by itself and not normally used as a typical ingredient of the food, whether or not it has nutritive value, the intentional addition of which to food is for a technical purpose, including an organoleptic purpose, in the manufacture, processing, preparation, treatment, packing, packaging, transport, or holding of such food. The use of a food additive results, or may be reasonably expected to result, (directly or indirectly) in it or its by-products becoming a component of or otherwise affecting the characteristics of the food. In a specific embodiment, a food additive of the invention can be added to a food resulting in a food composition of the invention.

In various embodiments, a food additive can be in solid form or liquid form. In one embodiment, a food additive of the invention can be incorporated into basic food ingredients, such as but not limited to syrups, starches, grains, flour, fats and oils, dietary fibers and bulking agents. Such food compositions fortified with a composition described herein can be used in the preparation of confectionery such as but not limited to biscuits, chocolates, candies, brownies, cookies, muffins; doughnuts, chewing gums, cakes, pies, ice creams and jellies; breads; pastas; noodles; processed soybean products such as tofu (bean curd); dairy products such as but not limited to yoghurt and butter; processed meat products such as but not limited to ham, hamburgers, and sausage; processed egg products such as tamago-yaki and egg custard; processed seafood based products such as ground fish meat products and imitation crab meat; seasonings such as sauce, dressing, mayonnaise and furikake (rice topping); dried fruits; cereals; pizzas; instant noodles; soups; snacks (e.g., chips, pretzels); and nutrition supplements such as food bars, sports bars, energy bars and the like. A food additive of the invention can also be added to ingredients used in food preparation, such as but not limited to, cooking oil, frying oil, salad oil, margarine, mayonnaise or peanut butter. A food additive of the invention can also be added to other foodstuffs such as but not limited to single cell protein, protein concentrates and isolates prepared from plants, algae, plant cell cultures, microorganisms, and animals, leaf meals, seed meals, concentrates and isolates from soybean, cottonseed, peanut, fish meal, and concentrates from meat, organs, and/or bones. The food additives of the invention can be used by the food industry to fortify bulk food ingredients or to prepare food products, or by consumers during food preparation. Any methods known to those skilled in the art may be used to add to or incorporate the compositions or compounds into natural or processed foodstuff to make the food composition of the invention. According to the invention, food compositions comprising one or more compound(s) or composition(s) of the invention are encompassed.

In another embodiment, the food additives of the invention can be used to prepare water-based food compositions. Oils containing the food additives of the invention can be emulsified and used in a variety of drinks. Accordingly, a food composition comprising compositions described herein can be a beverage, such as but not limited to fortified mineral water, fortified distilled water, a fruit juice-based beverage, a shake, a carbonated beverage, a lactic acid beverage, a sport beverage, milk, a milk-based beverage, a dairy product-based beverage, a yoghurt-based beverage, a carbonated water-based beverage, an alcoholic drink, a coffee-based beverage, a tea-based beverage, a green tea-based beverage, a black tea-based beverage, a grain-based beverage, a soybean-based beverage, soya-milk, an aloe-based beverage, or a beverage based on plant extracts. In a specific embodiment, a food additive or food composition of the invention can be a reconstitutable powder that, when reconstituted with a liquid, such as drinking water, can provide a beverage. Beverages comprising a composition(s) described herein are encompassed.

The compositions described herein can also be added to other food additives. Other food additives which can fortified with compositions described herein include but are not limited to natural sweeteners, artificial sweeteners, acidulants, anticaking agents, antioxidants, coloring agents, curing and pickling agents, emulsifiers, enzymes, fat replacers, firming agents, natural flavors, artificial flavors, flavor enhancers, humectants, leavening agents, lubricants, preservatives, stabilizers and thickeners. Conventional food additives enhanced with the compositions described herein are encompassed in the invention. Examples of food additives acceptable in manufacturing foods and beverages of the invention include sweeteners such as sucrose, glucose, fructose, isomerized liquid sugars, fructoligosaccharide, aspartame, sorbitol and stevia; coloring agents such as red cabbage colorant, grape pericarp colorant, elderberry colorant, caramel, gardenia colorant, corn colorant, saffron colorant and carotene; preservatives such as pectin decomposition products, benzoic acid, sorbic acid, parabens and potassium sorbate; thickeners such as sodium alginate, propylene glycol alginate, calcium cellulose glycolate and sodium cellulose glycolate; antioxidants such as L-ascorbic acid, tocopherol, erythrobic acid and rutin; color developing agents such as ferrous sulfate, sodium nitrite and potassium nitrate; bleaching agents such as sodium hydrogen nitrite and potassium metabisulfite; quality-keeping agents such as propylene glycol; quality improving agents such as L-cysteine hydrochloride and calcium stearyl lactate; inflating agents such as ammonium chloride, potassium hydrogen D-tartrate, ammonium carbonate, potassium carbonate, sodium hydrogen carbonate and alum; emulsifiers such as lecithin, sphingo-lipids, vegetable sterols, soybean saponin, sodium alginate, propylene glycol alginate, casein sodium, glycerol fatty acid esters, sucrose fatty acid esters and sorbitan fatty acid esters; emulsion stabilizers such as sodium chondroitin sulfate; flavoring substances such as lemon oil, eucalyptus oil, peppermint oil, vanilla extract, orange oil, garlic oil, ethyl acetoacetate, anisaldehyde, ethyl vanillin, cinnamic acid, citronellyl acetate, citral, vanillin, butyl butyrate and esters; nourishing agents such as L-ascorbic acid, L-asparagine, L-alanine, inositol, L-glutamine, carotene, tocopherol, vitamin A, folic acid, iron citrate, heme iron and uncalcined calcium; wheat flour-improving agents such as benzoyl peroxide, ammonium persulfate and chlorine dioxide; bactericides such as bleaching powder, hydrogen peroxide and hypochlorous acid; chewing gum bases such as methyl acetylricinolate, ester gum, vinyl acetate resin, polyisobutylene and polybutene; anti-blocking agents such as D-mannitol; integrating agents such as acidic sodium pyrophosphate, potassium pyrophosphate and sodium pyrophosphate; acidifiers such as adipic acid, citric acid, gluconic acid, succinic acid, D-tartaric acid, lactic acid and DL-malic acid; and seasonings such as fish extract, yeast extract, soy sauce, tomato puree, meat extract, mirin, fruit puree, dried bonito, sodium L-aspartate, DL-alanine, L-arginine L-glutamate, disodium 5′-inosinate, trisodium citrate, L-glutamic acid, sodium L-glutamate, succinic acid, L-tartaric acid and sodium lactate.

In another embodiment, the invention provides a dietary supplement comprising one or more compositions described herein. As used herein, the term “dietary supplement” means a product (other than tobacco) intended to supplement the diet that bears or contains one or more of the following dietary ingredients: a vitamin; a mineral; an herb or other botanical; an amino acid; a dietary supplement used by man to supplement the diet by increasing the total dietary intake; or a concentrate, metabolite, constituent, extract, or a combination of any of the ingredients. Typically, a dietary supplement is a product that is labeled as a dietary supplement and is not represented for use as a conventional food or as a sole item of a meal or the diet. A dietary supplement can be consumed by a subject independent of any food, unlike a food additive which is incorporated into a food composition during the processing, manufacture, preparation, or delivery of the food composition, or just before its consumption.

In yet another embodiment, the invention provides a medical food comprising one or more compositions described herein. As used herein, the term “medical food” refers to a food which is formulated to be consumed or administered enterally under the supervision of a physician and which is intended for the specific dietary management of a disease or condition for which distinctive nutritional requirements, based on recognized scientific principles, are established by medical evaluation. Examples of medical foods include, but are not limited to, sole source nutrition products which are complete nutritional products used to replace all other food intake; oral rehydration solutions for use in replacing fluids and electrolytes lost following diarrhea or vomiting; modular nutrient products containing specially selected components not intended to be complete nutritional sources but designed for the management of specific diseases and which have associated claims to effectiveness either direct or implied; and products intended for use in dietary management of inborn errors of metabolism.

In yet another embodiment, the invention provides a food for special dietary use comprising one or more compositions described herein. As used herein, the term “food for special dietary use” refers to a food which purports or is represented to be used, including but not limited to the following: supplying a special dietary need that exists by reason of a physical, physiological, pathological, or other condition, including but not limited to the condition of disease, convalescence, pregnancy, lactation, infancy, allergic hypersensitivity to food, underweight, overweight, or the need to control the intake of sodium; supplying a vitamin, mineral, or other ingredient for use by man to supplement his diet by increasing the total dietary intake; and supplying a special dietary need by reason of being a food for use as the sole item of the diet.

The compositions described herein can be included in a dietary supplement, medical food, or food for special dietary use, which also include one or more other ingredients that impart a healthful or medicinal benefit. The optional ingredients useful herein can be categorized by their healthful benefit or their postulated mode of action. However, it is to be understood that the optional components useful herein can in some instances provide more than one healthful benefit or operate via more than one mode of action. Therefore, classifications herein are made for the sake of convenience and are not intended to limit the component to that particular application or applications listed.

A dietary supplement, medical food, or food for special dietary use, of the invention can comprise in addition to one or more composition(s) described herein, one or more additional ingredient(s), such as but not limited to vitamins, minerals, electrolytes, sports nutritional products, amino acids, probiotics, metabolites, hormones, enzymes, cartilage products, botanical extracts, and homeopathic products. More specifically, a dietary supplement of the invention comprises one or more composition(s) described herein and one or more substance(s) from the following non-limiting categories: (i) amino acids and oligopeptides, such as but not limited to 5-hydroxytryptophan, acetyl-L-carnitine, acetylcysteine, arginine pyroglutamate, branched-chain amino acids, creatine, DL-phenylalanine (phenylalanine), dimethylglycine (DMG), glutamine peptides, glutathione, glycine, insulin-like growth factor 1, L-aspartate, L-carnitine, L-cysteine, L-glutamine, L-histidine, L-lysine (lysine), L-methionine (methionine), L-ornithine, L-phenylalanine (phenylalanine), L-theanine, L-tyrosine (tyrosine), lactoferrin, ornithine alpha-ketoglutarate, para-aminobenzoic acid (aminobenzoic acid), taurine; (ii) glycosupplements, such as but not limited to chitosan, D-glucarate, D-ribose, fructo-oligosaccharides, glucomannan, glucosamine, inulins (inulin), lactulose, larch arabinogalactan, modified citrus pectin, pectin, psyllium (psyllium husk), sodium alginates, yeast beta-D-glucans; (iii) hormones, such as but not limited to 19-norandrostenedione, androstenediol, androstanedione, beta-sitosterol, biochanin A, DHEA, glandulars, human growth hormone and secretagogues (somatropin), ipriflavone, pregnenolone, soy isoflavones, tiratricol (TRIAC); lipids such as but not limited to alkoxyglycerols, blackcurrant seed oil, borage oil, caprylic acid, cetyl myristoleate, conjugated linoleic acid (CLA), docahexaenoic acid (DHA), eicosapentaenoic acid (EPA), evening primrose oil, flaxseed oil, glycerol (glycerin), hemp seed oil, hexacosanol, inositol hexaphosphate, L-alpha-glycerylphosphorylcholine (Alpha-GPC), lithium gamma-linolenic acid (Li-GLA), medium-chain triglycerides, myo-inositol, octacosanol, perilla oil, phosphatidylcholine, phosphatidylserine, policosanol, squalene, plant stanols; (iv) metabolites and cofactors such as but not limited to 7-oxo-dehydroepiandrosterone, alpha-lipoic acid, betaine and betaine hydrochloride, CDP-choline (citicolin sodium), coenzyme Q10 (CoQ10), NADH, pantethine, and pyruvate; (v) minerals and electrolytes, such as but not limited to metal salts, chelated minerals, colloidal minerals, colloidal silver, colloidal gold, bentonite, compounds comprising aluminum, arsenic, bromine, calcium, chromium, copper, fluoride, germanium, iodine, iron, lithium, magnesium, manganese, molybdenum, nickel, phosphorus, potassium, selenium, selenium, silicon, tin, vanadium, and zinc; (vi) mycosupplements such as but not limited to brewer's yeast , kombucha, myco-polysaccharides, and red yeast rice; (vii) inosine, nucleic acids, nucleotides; (viii) microorganisms such as but limited to prebiotics, probiotics, synbiotics, and yogurt organisms; (ix) proteins such as but not limited to bovine cartilage, bovine colostrum, bromelain (bromelains), chicken collagen II, gelatin hydrolysates (gelatin), hydrolyzed collagen, shark cartilage, soy protein, and whey proteins; (x) vitamins in either natural or synthetic form, such as but are not limited to, vitamin A (e.g., beta carotene, retinoic acid, retinol, retinoids, retinyl palmitate, retinyl proprionate, etc.), vitamin B (e.g., niacin, niacinamide, riboflavin, pantothenic acid, etc.), vitamin B6 (pyridoxine hydrochloride), vitamin B12 (cyanocobalamin), vitamin C (e.g., ascorbic acid, etc.), vitamin D (e.g., ergosterol, ergocalciferol, cholecalciferol, etc.), Vitamin E (e.g., tocopherol acetate, etc.), vitamin K (e.g., phytonadione, menadione, phthiocol, etc.), alpha-tocopheryl nicotinate, alpha-tocopheryl polyethylene glycol succinate, ascorbyl palmitate, biotin, folate (folio acid), gamma-tocopherol, inositol nicotinate (inositol niacinate), niacin, nicotinamide (niacinamide), pantothenic acid (calcium pantothenate), thiamin, and tocotrienols; (xi) botantical extracts such as DHEA, Ginkgo biloba extracts, ginseng extracts, and reisi (Ganoderma) extract; and (xii) other supplements known in the art such as but not limited to activated charcoal, beta-hydroxy-beta-methylbutyrate (HMB), choline, colosolic acid, deanol, dimethyl sulfoxide (DMSO), dolomite, gamma-butyrolactone (GBL), gamma-hydroxybutyrate (GHB), liver hydrolysate/desiccated liver, malic acid, methylsulfonylmethane (MSM), royal jelly, vinpocetine, arnica, bee pollen, chlorella, chlorophyll/chlorophyllin (chlorophyllin copper complex), chrysin, cocoa flavonoids, curcuminoids, daidzein, deglycyrrhizinated licorice (DGL), flower pollen, genistein, glycitein, grape seed proanthocyanidins, green tea catechins, hesperetin, hesperidin, huperzine A, hydroxycitric acid, hydroxyethylrutosides, indole-3-carbinol, lutein and zeaxanthin, lycopene, oat beta-D-glucan, phytostanols, phytosterols, piperine, propolis, pycnogenol, quercetin, resveratrol, rutin, secoisolariciresinol diglycoside (SDG), soy isoflavones, spirulina, sulforaphane, and wheat grass/barley grass.

Non-limiting examples of minerals and electrolytes include but is not limited to calcium compounds, calcium carbonate, calcium citrate, iron compounds, iron fumarate, iron gluconate, iron sulfate, magnesium compounds, magnesium carbonate, magnesium chloride, magnesium gluconate, selenium compounds, and manganese compounds.

Also encompassed by the invention are nutraceutical compositions comprising one or more compound(s) or composition of the invention and one or more “Generally Regarded As Safe” (“GRAS”) substance(s). Many GRAS substances are known and are listed in the various sections of the regulations of the United States public health authority, 21 CFR 73, 74, 75, 172, 173, 182, 184 and 186, which are incorporated herein by reference in their entirety.

For example, the following GRAS flavor alcohols can be used in combination with the compounds and compositions of the invention, for example: benzyl alcohol, acetoin (acetylmethylcarbinol), ethyl alcohol (ethanol), propyl alcohol (1-propanol), iso-propyl alcohol (2-propanol, isopropanol), propylene glycol, glycerol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), .alpha.-methylbenzyl alcohol (1-phenyl-ethanol), heptyl alcohol (heptanol), n-amyl alcohol (1-pentanol), iso-amyl alcohol (3-methyl-l-butanol), anisalcohol (4-methoxybenzyl alcohol, p-anisalcohol), citronellol, n-decyl alcohol (n-decanol), geraniol, beta-gamma-hexanol (3-hexenol), lauryl alcohol (dodecanol), linalool, nerolidol, nonadienol (2,6-nonadiene-1-ol), nonyl alcohol (nonanol-1), rhodinol, terpineol, borneol, clineol (eucalyptol), anisole, cuminyl alcohol (cuminol), 10-undecen-1-ol, 1-hexadecanol. Suitable derivatives include, for example, the esters, ethers and carbonates of the above mentioned GRAS flavor alcohols are contemplated. Particularly preferred GRAS flavor alcohols are benzyl alcohol, 1-propanol, glycerol, propylene glycol, n-butyl alcohol, citronellol, hexanol, linalool, acetoin and their derivatives.

Also encompassed is the inclusion of one or more GRAS polyphenols in the nutraceutical compositions of the invention, such as but not limited to catechol, resorcinol, hydroquinone, phloroglucinol, pyrogallol, cyclohexane, usnic acid, acylpolyphenols, lignins, anthocyans, flavones, catechols, gallic acid derivatives (e.g., tannins, gallotannin, tannic acids, gallotannic acids), catechins, theaflavins, camosol, camosolic acid (including their derivatives, such as (2,5-dihydroxyphenyl)carboxylic and (2,5-dihydroxyphenypalkylenecarboxylic substitutions, salts, esters, amides), caffeic acid and its esters and amides, flavonoids (e.g., flavone, flavonol, isoflavone, gossypetin, myricetin, robinetin, apigenin, morin, taxifolin, eriodictyol, naringin, rutin, hesperidin, troxerutin, chrysin, tangeritin, luteolin, catechols, quercetin, fisetin, kaempferol, galangin, rotenoids, aurones, flavonols, diols), extracts, e.g., from Camellia (C. sinensis in particular), Primula. Further, their possible derivatives, e.g., salts, acids, esters, oxides and ethers, may also be used.

Also encompassed is the inclusion of one or more GRAS acids in the nutraceutical compositions of the invention, such as but not limited to acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether), phenylacetic acid (.alpha.-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid, tannic acid and their derivatives. Suitable derivatives According to the present invention are esters (e.g., C.sub.1-6-alkyl esters and benzyl esters), amides (including N-substituted amides) and salts (alkali, alkaline earth and ammonium salts) of the above mentioned acids. According to the present invention, the term “derivatives” also encompasses modifications of the side-chain hydroxy functions (e.g., acyl and alkyl derivatives) and modifications of the double bonds (e.g., the perhydrogenated and hydroxylated derivatives of the mentioned acids).

Also encompassed is the inclusion of one or more GRAS phenols in the nutraceutical compositions of the invention, such as but not limited to thymol, methyleugenol, acetyleugenol, safrol, eugenol, isoeugenol, anethole, methylchavicol (estragol; 3-(4-methoxyphenyl)-1-propene), carvacrol, alpha-bisabolol, fornesol, anisole (methoxybenzene), propenylguaethol (5-propenyl-2-ethoxyphenol) and their derivatives. Derivatives according to the present invention are compounds in which the phenolic hydroxy group has been esterified or etherified.

Also encompassed is the inclusion of one or more GRAS esters in the nutraceutical compositions of the invention, such as but not limited to allicin and the following acetates may be used, for example: iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate, hexyl acetate (hexanyl ethanoate), hydrocinnamyl acetate (3-phenylpropyl acetate), linalyl acetate, octyl acetate, phenylethyl acetate, terpinyl acetate, triacetin (glyceryl triacetate), potassium acetate, sodium acetate and calcium acetate.

Also encompassed is the inclusion of one or more GRAS terpenes in the nutraceutical compositions of the invention, such as but not limited to camphor, limonene and beta-caryophyllene. Also encompassed is the inclusion of one or more GRAS acetals in the nutraceutical compositions of the invention, such as but not limited to acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal and benzaldehyde propylene glycol acetal. Also encompassed is the inclusion of one or more GRAS acetaldehydes in the nutraceutical compositions of the invention, such as but not limited to acetaldehyde, anisaldehyde, benzaldehyde, iso-butyl aldehyde (methyl-1-propanal), citral, citronellal, n-caprylic aldehyde (n-decanal), ethylvanillin, furfural, heliotropin (piperonal), heptyl aldehyde (heptanal), hexyl aldehyde (hexanal), 2-hexenal (beta-propylacrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde and cuminaldehyde.

Also encompassed is the inclusion of one or more GRAS essential oils in the nutraceutical compositions of the invention, such as but not limited to essential oils and/or alcoholic or glycolic extracts or extracts obtained by high-pressure carbon-dioxide processes from plants such as : oils or extracts having a high content of alcohols: melissa, coriander, cardamon, eucalyptus; oils or extracts having a high content of aldehydes: Eucalyptus citriodora, cinnamon, lemon, lemon grass, melissa, citronella, lime, orange; oils or extracts having a high content of phenols: origanum, thyme, rosemary, orange, clove, fennel, camphor, mandarin, anise, cascarilla, estragon and pimento; oils or extracts having a high content of acetates: lavender; oils or extracts having a high content of esters: mustard, onion, garlic; oils or extracts having a high content of terpenes: pepper, bitter orange, caraway, dill, lemon, peppermint, nutmeg; oils or extracts having a high content of acids: olibanum.

Any of the additional substances in a nutraceutical composition of the invention may be included as the substantially pure material, or as an extract obtained by suitable physical and/or chemical isolation from natural (e.g., plant) sources.

In certain embodiments, the meaning of the term “medical food”, “food for special dietary use”, “dietary supplement” or “food additive” is the meaning of those terms as defined by a regulatory agency of the Federal or a state government, including the United States Food and Drug Administraion.

In certain embodiments, the nutraceutical compositions of the present invention comprise from about 0.001% to about 98%, preferably about 25% to about 98%, more preferably about 40% to about 98% by weight of a composition described herein. Other amounts of the combination that are also contemplated are from about 0.01% to about 35%, 0.1% to about 20%, 0.1% to about 15%, 1% to about 10%, and 2% to about 7%, by weight of a composition described herein.

Cosmetic Compositions

In another embodiment, the present invention provides cosmetic compositions comprising compositions described herein. Also included is a nonexclusive description of various optional and preferred components useful in embodiments of the present invention. When used in the context of a cosmetic composition, the terms “safe and effective amount” means an amount of a compound, component, or composition (as applicable) sufficient to significantly induce a positive effect (e.g., confer a noticeable cosmetic benefit), but low enough to avoid serious side effects, (e.g., undue toxicity or allergic reaction), i.e., to provide a reasonable benefit to risk ratio, within the scope of sound medical judgment.

The cosmetic composition of the present invention is suitable for providing healthful, therapeutic and aesthetic skin benefits by contacting, deposition and/or adhesion to skin and/or hair, or by providing and maintaining body and/or hair hygiene. Suitable cosmetic agents include, but are not limited to those selected from the group consisting of absorbents, anti-acne agents, anti-caking agents, anti-cellulite agents, anti-foaming agents, anti-fungal agents, anti-inflammatory agents, anti-microbial agents, antioxidants, antiperspirant/deodorant agents, anti-skin atrophy agents, antiviral agents, anti-wrinkle agents, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin detergents, skin thickeners, sunscreen agents, topical anesthetics, vitamins, and combinations thereof.

The cosmetic compositions of the present invention may also comprise a cosmetically-acceptable carrier and any optional components. Suitable carriers are well known in the art and are selected based on the end use application. For example, carriers of the present invention include, but are not limited to, those suitable for application to skin. Preferably, the carriers of the present invention are suitable for application to skin (e.g., sunscreens, creams, milks, lotions, masks, serums, etc.) and nails (e.g., polishes, treatments, etc.). Such carriers are well-known to one of ordinary skill in the art, and can include one or more compatible liquid or solid filler diluents or vehicles which are suitable for application to skin and nails. The exact amount of carrier will depend upon the level of the bonding agent and any other optional ingredients that one of ordinary skill in the art would classify as distinct from the carrier (e.g., other active components). The compositions preferably comprise from about 25% to about 99.999%, more preferably from about 40% to about 99.99%, still more preferably from 75% to about 99%, and most preferably, from about 93% to about 98%, by weight of the composition, of a carrier.

The carrier and compositions herein can be formulated in a number of ways, including but not limited to emulsions (in emulsion technology, a composition comprising a “dispersed phase” and a “continuous phase;” the dispersed phase existing as small particles or droplets that are suspended in and surrounded by a continuous phase). For example, suitable emulsions include oil-in-water, water-in-oil, water-in-oil-in-water, oil-in-water-in-oil, and oil-in-water-in-silicone emulsions. Preferred compositions comprise an oil-in-water emulsion.

The cosmetic compositions of the present invention can be formulated into a wide variety of product types, including creams, waxes, pastes, lotions, milks, mousses, gels, oils, tonics, and sprays. Preferred compositions are formulated into lotions, creams, gels, and sprays. These product forms may be used for a number of applications, including, but not limited to, soaps, shampoos, hair, hand and body lotions, cold creams, facial moisturizers, anti-acne preparations, topical analgesics, make-ups/cosmetics including foundations, eyeshadows, lipsticks, and the like. Any additional components required to formulate such products vary with product type and can be routinely chosen by one skilled in the art.

If compositions of the present invention are formulated as an aerosol and applied to the skin as a spray-on product, a propellant is added to the composition. Examples of suitable propellants include chlorofluorinated lower molecular weight hydrocarbons. A more complete disclosure of propellants useful herein can be found in Sagarin, Cosmetics Science and Technology, 2nd Edition, Vol. 2, pp. 443-465 (1972).

The compositions may contain a variety of other components such as are conventionally used in a given product type provided that they do not unacceptably alter the benefits of the invention. These optional components should be suitable for application to mammalian skin, that is, when incorporated into the compositions they are suitable for use in contact with human skin without undue toxicity, incompatibility, instability, allergic response, and the like, within the scope of sound medical or formulator's judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: enzymes, surfactants, abrasives, skin exfoliating agents, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, polymer beads, film formers, fragrances, humectants, opacifying agents, pH adjusters, propellants, reducing agents, sequestrants, skin bleaching agents (or depigmenting, lightening agents) (e.g., hydroquinone, azelaic acid, caffeic acid, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, bisabolol, and dipotassium glycyrrhizinate), thickeners, hydrocolloids, particular zeolites, and vitamins and derivatives thereof (e.g., tocopherol, tocopherol acetate, beta carotene, retinoic acid, retinol, retinoids, retinyl palmitate, niacin, niacinamide, and the like).

Further examples of optional components include wetting agents; emollients; moisturizing agents such as glycerol, PEG 400, thiamorpholinone and derivatives thereof, or urea; anti-seborrhoea agents such as S-carboxymethylcysteine, S-benzylcysteamine, the salts and the derivatives thereof; antibiotics such as erythromycin and esters thereof, neomycin, clindamycin and esters thereof, and tetracyclines; antifungal agents such as ketoconazole or 4,5-polymethylene-3-isothiazolidones; agents for promoting the regrowth of the hair, such as minoxidil (2,4-diamino-5-piperidinopyridine 3-oxide) and derivatives thereof, diazoxide (7-chloro-3-methyl-1,2,4-benzothiadiazine 1,1-dioxide) and phenytoin (5,4-diphenylimidazolidine-2,4-dione); non-steroidal anti-inflammatory agents; carotenoids and, in particular, b-carotene; anti-psoriatic agents such as anthralin and derivatives thereof. The cosmetic compositions according to the invention may also contain flavor-enhancing agents, preserving agents such as para-hydroxybenzoic acid esters, stabilizing agents, moisture regulators, pH regulators, osmotic pressure modifiers, emulsifying agents, UV-A and UV-B screening agents, and antioxidants such as butylhydroxyanisole or butylhydroxytoluene.

The compositions of the present invention may include carrier components such as are known in the art. Such carriers can include one or more compatible liquid or solid filler diluents or vehicles that are suitable for application to skin and/or hair.

Other optional components in a food additive of the invention include but are not limited to anti-caking agent, dessicant, food preservatives, food coloring, and artificial sweetener.

Pharmaceutical Compositions

The compositions of the invention include bulk-drug compositions (which can be non-sterile) useful in the manufacture of pharmaceutical compositions and in the preparation of unit dosage forms. In one embodiment, a composition described herein is a pharmaceutical composition or a single unit dosage form (and is preferably sterile). Pharmaceutical compositions and single unit dosage forms of the invention comprise a prophylactically or therapeutically effective amount of one or more compositions described herein, and a typically one or more vehicles, carriers, or excipients. Preferably, the vehicles, carriers, or excipients are pharmaceutically acceptable. The term “pharmaceutically acceptable” means approved by a regulatory agency of the Federal or a state government or listed in the U.S. Pharmacopeia or other generally recognized pharmacopeia for use in animals, and more particularly in humans.

This invention further encompasses anhydrous pharmaceutical compositions and dosage forms. Anhydrous pharmaceutical compositions and dosage forms of the invention can be prepared using anhydrous or low moisture containing ingredients and low moisture or low humidity conditions. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The term “vehicle” refers to a diluent, adjuvant, excipient, carrier, or filler with which the compound or composition of the invention is stored, transported, and/or administered. Suitable vehicles are well-known to those skilled in the art of pharmacy, and non-limiting examples of suitable vehicles include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Such pharmaceutical vehicles can be sterile liquids, such as water and oils, including those of petroleum, animal, vegetable or synthetic origin, such as peanut oil, soybean oil, mineral oil, sesame oil and the like. Water is a preferred vehicle when the pharmaceutical composition is administered intravenously. Saline solutions and aqueous dextrose and glycerol solutions can also be employed as liquid vehicles, particularly for injectable solutions. Whether a particular vehicle is suitable for incorporation into a pharmaceutical or nutraceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

The invention further encompasses pharmaceutical compositions and dosage forms that comprise one or more compounds that reduce the rate by which an active ingredient will decompose. Such compounds, which are referred to herein as stabilizers, include, but are not limited to, antioxidants such as ascorbic acid, pH buffers, or salt buffers.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra-synovial and rectal administration. In various embodiments, the pharmaceutical compositions or single unit dosage forms are sterile and in suitable form for administration to a subject, preferably an animal subject, more preferably a mammalian subject, and most preferably a human subject.

The composition, shape, and type of dosage forms will typically vary depending on their use. Examples of dosage forms include, but are not limited to: tablets; caplets; capsules, such as soft elastic gelatin capsules; pills; pellets; capsules containing liquids cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. Formulations in the form of powders or granulates may be prepared using the ingredients mentioned above under tablets and capsules in a conventional manner using, e.g., a mixer, a fluid bed apparatus or a spray drying equipment.

Generally, a dosage form used in the acute treatment of a condition associated with inflammation (e.g., an inflammatory disorder) may contain larger amounts of one or more of the active ingredients it comprises than a dosage form used in the chronic treatment of the same condition. Also, the prophylactically and therapeutically effective dosage form may vary among different types of conditions associated with inflammation (e.g., an inflammatory disorder). Similarly, a parenteral dosage form may contain smaller amounts of one or more of the active ingredients it comprises than an oral dosage form used to treat the same condition. These and other ways in which specific dosage forms encompassed by this invention will vary from one another and will be readily apparent to those skilled in the art. See, e.g., Remington's Pharmaceutical Sciences, Gennaro, et al., 19th Ed., Easton, Pa., Mack Publishing Co., (1995); Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988.

The invention also provides that a pharmaceutical composition can be packaged in a hermetically sealed container such as an ampoule or sachette indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient. In another embodiment, the pharmaceutical composition is supplied in an aqueous solution, such as water or saline, in a hermetically sealed container. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Oral Dosage Forms

The present invention provides pharmaceutical compositions that are suitable for oral administration, as well as other orally comsumable compositions, including but not limited to nutraceutical compositions and in particular, dietary supplements. Such oral compositions can be presented as discrete dosage forms, such as, but are not limited to, tablets (e.g., chewable tablets), caplets; capsules, such as soft elastic gelatin capsules; pills; pellets; capsules containing liquids cachets; troches; lozenges; suspensions (e.g., aqueous or non-aqueous liquid suspensions, oil-in-water emulsions, or a water-in-oil liquid emulsions), solutions, and elixirs. Such dosage forms contain predetermined amounts of active ingredients, and may be prepared by methods of pharmacy well known to those skilled in the art. See generally, Remington's Pharmaceutical Sciences, Gennaro, et al., 19th Ed., Easton, Pa., Mack Publishing Co., (1995) and Remington: The Science and Practice of Pharmacy by Gennaro (2000).

Typical oral dosage forms of the invention are prepared by combining the active ingredient(s) in an intimate admixture with at least one vehicle excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Formulations for oral use may also be presented as chewing tablets, or as hard gelatin capsules wherein the active ingredient is mixed with an inert solid diluent, for example, potato starch, lactose, microcrystalline cellulose, calcium carbonate, calcium phosphate or kaolin, or as soft gelatin capsules wherein the active ingredient is mixed with water or an oil medium, for example, peanut oil, liquid paraffin, or olive oil.

Liquid preparations for oral administration can take the form of, for example, solutions, syrups or suspensions, or they can be presented as a dry product for constitution with water or other suitable vehicle before use. Such liquid preparations can be prepared by conventional means with vehicles such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

In various embodiments, many excipients known in the art can be used in oral dosage forms of the invention include, but are not limited to, binders, fillers, disintegrants, lubricants, dispersing agent, wetting agent, and suspending agent. Binders suitable for use in pharmaceutical/nutraceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions, dietary supplements, and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM. The binder or filler in pharmaceutical compositions of the invention can be present in from about 50 to about 99 weight percent of the pharmaceutical composition, nutraceutical composition, dietary supplement, or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions and dietary supplement comprise from about 0.5 to about 15 weight percent of disintegrant, specifically from about 1 to about 5 weight percent of disintegrant.

Disintegrants that can be used in pharmaceutical compositions, dietary supplements and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions, dietary supplmenents, and dosage forms of the invention include, but are not limited to, calcium stearate, magnesium stearate; thfileral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions, dietary supplements, or dosage forms into which they are incorporated.

Suitable dispersing or wetting agents are, for example, naturally-occurring phosphatides, as e.g., lecithin, or condensation products of ethylene oxide with e.g., a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc.

Parenteral Dosage Forms

The present invention provides parenteral dosage forms of the compositions described herein. Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Typically, compositions for intravenous administration are solutions in sterile isotonic aqueous buffer.

Compounds that increase the solubility of one or more of the active ingredients disclosed herein can also be incorporated into the parenteral dosage forms of the invention. Where necessary, the composition may also include a solubilizing agent and a local anesthetic such as lignocamne to ease pain at the site of the injection.

Transdermal, Topical & Mucosal Dosage Forms

The present invention provides transdermal, topical and/or mucosal dosage forms of the compositions described herein. Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, Gennaro, et al., 19th Ed., Easton, Pa., Mack Publishing Co., (1995); Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical and cosmetic arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Emulsifying agents, preservatives, antioxidants, gel-forming agents, chelating agents, moisturizers or humectants can also be added to pharmaceutical compositions and dosage forms if desired. Examples of such additional ingredients are well known in the art. See, e.g., Remington's Pharmaceutical Sciences, Gennaro, et al., 19th Ed., Easton, Pa., Mack Publishing Co., (1995); Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999).

Examples of emulsifying agents are naturally occurring gums, e.g. gum acacia or gum tragacanth, naturally occurring phosphatides, e.g. soybean lecithin and sorbitan monooleate derivatives. Examples of antioxidants are butylated hydroxy anisole (BHA), ascorbic acid and derivatives thereof, tocopherol and derivatives thereof, butylated hydroxy anisole and eysteine. Examples of preservatives are parabens, such as methyl or propyl p-hydroxybenzoate and benzalkonium chloride. Examples of humectants are glycerin, propylene glycol, sorbitol and urea. Examples of chelating agents are sodium EDTA, citric acid and phosphoric acid. Examples of gel forming agents are Carbopol, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

In a specific embodiment, the invention provides formulations for administration to the eye in the form of eye drops, lotions, ointments or delivery devices. Typically, the composition comprises the active compound(s) in combination with vehicles or the active compound is incorporated in a suitable carrier system. Pharmaceutically inert vehicles and/or excipients for the preparation of eye drops include, e.g., buffering agents such as boric acid or borates, pH adjusting agents to obtain optimal stability or solubility of the active compound, tonicity adjusting agents such as sodium chloride or borates, viscosity adjusting agents such as hydroxypropyl cellulose, methylcellulose, polyvinylpyrrolidone, polyvinyl alcohols or polyacrylamide, oily vehicle such as vehicles comprising arachis oil, castor oil and/or mineral oil. Emulsions and suspensions of the active drug substance may also be presented in form of eye drops. In these cases, the composition may furthermore comprise stabilizing, dispersing, wetting, emulsifying and/or suspending agents.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Controlled Release Dosage Forms

The invention also provides controlled release dosage forms comprising a composition described. As used herein, the terms “controlled release dosage form” and “controlled release formulation” are used interchangeably to refer to (i) formulations which create a substantially constant concentration of the drug within the body of a subject over an extended period of time, (ii) formulations which after a predetermined lag time create a substantially constant concentration of the drug within the body over an extended period of time, (iii) formulations which sustain drug action during a predetermined time period by maintaining a relatively, constant, effective drug level in the body with concomitant minimization of undesirable side effects associated with fluctuations in the plasma level of the active drug substance (e.g., a sawtooth kinetic pattern), (iv) formulations which attempt to localize drug action by, e.g., spatial placement of a controlled release composition adjacent to or in the diseased tissue or organ, (v) formulations which attempt to target drug action by using carriers or chemical derivatives to deliver the drug to a particular target cell type. Controlled release formulations are also known in the art as “sustained release”, “prolonged release”, “programmed release”, “time release”, “rate-controlled” and/or “targeted release” formulations.

Compositions intended to be administered by controlled release means may be presented in any suitable dosage forms, especially in dosage forms intended for oral, parenteral, cutaneous nasal, rectal, vaginal and/or ocular administration. Such dosage forms can be used to provide controlled-release of one or more active ingredients using, for example, polymer matrices, gels, permeable membranes, osmotic systems, multilayer coatings, microparticles, nanoparticles, liposomes, microspheres, or a combination thereof to provide the desired release profile in varying proportions. Suitable controlled-release formulations known to those of ordinary skill in the art, including those described herein, can be readily selected for use with the compounds and compositions of the invention. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference.

In one embodiment, the invention encompasses single unit dosage forms suitable for oral administration such as, but not limited to, tablets, capsules, gelcaps, and caplets that are adapted for controlled-release. The dosage forms can be uncoated or they can be coated by known techniques to delay disintegration and absorption in the gastrointestinal tract and thereby providing a sustained action over a longer period. The coating can be adapted to release the active drug substance in a predetermined pattern, e.g, in order to achieve a controlled release formulation or it can be adapted not to release the active drug substance until after passage of the stomach (enteric coating). The coating can be a sugar coating, a film coating (e.g., based on hydroxypropyl mothylcellulose, methylcellulose, methyl hydroxyethylcellulose, hydroxypropylcellulose, carboxymethylcellulose, acrylate copolymers (Eudragit EO), polyethylene glycols and/or polyvinylpyrrolidone) or an enteric coating (e.g., based on methacrylic acid copolymer (Eudragit* L and S), cellulose acetate phthalate, hydroxypropyl methylcellulose phthalate, hydroxypropyl metbylcellulose acetate succinate, polyvinyl acetate phthalate, shellac and/or ethylcellulose). A time delay material such as, e.g., glyceryl monostearate or glyceryl distearate can also be employed.

In a specific embodiment, a buoyant tablet formulation of a composition described herein can be prepared by granulating a mixture of the compound or composition, excipients and 20-75% w/w of hydrocolloids, such as hydroxyethylcellulose, hydroxypropylcellulose and hydroxypropylmethylcellulose. The obtained granules can then be compressed into tablets. On contact with the gastric juice, the tablet can form a substantially water-impermeable gel barrier around its surface. This gel barrier takes part in maintaining a density of less than one, thereby allowing the tablet to remain buoyant in the gastric juice.

All controlled-release pharmaceutical products and dietary supplements have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient complaint. In addition, controlled-release formulations can be used to affect the time of onset of action or other characteristics, such as blood levels of the drug, and can thus affect the occurrence of side (e.g., adverse) effects.

Accordingly, the use of controlled release dosage forms of the invention is especially preferred in such cases where a compound or composition of the invention (i) has a narrow therapeutic index, i.e., the difference between the plasma concentration leading to harmful side effects or toxic reactions and the plasma concentration leading to a therapeutic effect is small; in general, the therapeutic index, TI, is defined as the ratio of median lethal dose (LD50) to median effective dose (ED50), (ii) has a narrow absorption window in the gastro-intestinal tract, (iii) has a very short biological half-live so that frequent dosing during a day is required in order to sustain the plasma level at a therapeutic level, (iv) is desired to be used only once or twice daily or even less frequent with the purpose of reducing patient compliance problems, v) is desired to be present in plasma without peak concentrations that is harmful or at a minimally fluctuating concentration in plasma.

Many controlled-release formulations are designed to initially release an amount of a composition that promptly produces the desired therapeutic or prophylactic effect, and gradually and continually release of other amounts of the composition to maintain this level of therapeutic or prophylactic effect over an extended period of time. In order to maintain this constant level of drug in the body, a composition must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Prophylactic and Therapeutic Methods

The present invention provides methods for preventing, reducing, or eliminating the symptoms and conditions associated with inflammation, the methods comprising administering to a subject a theaflavin composition and one or more therapies other than such a theaflavin composition. Preferably, such therapies are currently being used, have been used or are known to be useful in the prevention, treatment, management and/or amelioration of a condition associated with inflammation (e.g., an inflammatory disorder) or a symptom thereof. Sections 4.2 and 4.3, supra, provides non-limiting examples of the therapies which can be used in combination with a theaflavin composition for the prevention, treatment, management and/or amelioration of a condition associated with inflammation (e.g., an inflammatory disorder) or a symptom thereof. In a specific embodiment, the present invention provides a method of preventing, treating, managing, or ameliorating a condition associated with inflammation (e.g., an inflammatory disorder) or one or more symptoms thereof, said method comprising administering to a subject in need thereof a prophylactically or therapeutically effective amount of a theaflavin composition of the invention and a prophylactically or therapeutically effective amount of one or more other therapies (e.g., one or more other prophylactic or therapeutic agents). In a preferred embodiment, the administration of a theaflavin composition and one or more other therapies has a syngeristic effect.

The invention provides methods for preventing, managing, treating and/or ameliorating a condition associated with inflammation (e.g., an inflammatory disorder) or one or more symptoms thereof in a subject refractory to existing single agent therapies for such a condition, the methods comprising administering to said subject a prophylactically or therapeutically effective amount of a theaflavin composition of the invention and a prophylactically or therapeutically effective amount of one or more other therapies (e.g., one or more other prophylactic or therapeutic agents). The invention also provides methods for preventing, treating, managing, and/or ameliorating a condition associated with inflammation (e.g., an inflammatory disorder), the methods comprising administering a prophylactically or therapeutically effective amount of a theaflavin composition of the invention and a prophylactically or therapeutically effective amount of any other therapy(ies) to subjects who have proven refractory to other therapies but are no longer on these therapy(ies). The present invention also provides alternative methods for the prevention, treatment, management, and/or amelioration of a condition associated with inflammation (e.g., an inflammatory disorder) where another therapy has proven or may prove too toxic, i.e., results in unacceptable or unbearable side effects, for the subject being treated. Further, the present invention provides methods for preventing the recurrence of a condition associated with inflammation (e.g., an inflammatory disorder) in subjects that have been treated and have no disease activity, the methods administering to the subject a prophylactically or therapeutically effective amount of a theaflavin composition of the invention and a prophylactically or therapeutically effective amount of one or more other therapies (e.g., one or more other prophylactic or therapeutic agents).

Examples of the inflammatory disorders which can be prevented, managed, treated, and/or ameliorated in accordance with the methods of the invention, include, but are not limited to, asthma, allergic reactions, allergic disorders, inflammatory disorders characterized by type-1 mediated inflammation, inflammatory disorders characterized by type-2 mediated inflammation, fibrotic disease (e.g., pulmonary fibrosis), psoraisis, multiple sclerosis, systemic lupus erythrematosis, chronic obstructive pulmonary disease (COPD), encephilitis, inflammatory bowel disease (e.g., Crohn's disease and ulcerative colitis), ischemic reperfusion injury, Gout, Behcet's disease, septic shock, undifferentiated spondyloarthropathy, undifferentiated arthropathy, arthritis, reactive arthritis, rheumatoid arthritis (juvenile and adult), osteoarthritis, psoriatic arthritis, inflammatory osteolysis, degenerative joint diseases, sepsis, meningitis, and chronic inflammation resulting from, e.g., chronic viral or bacteria infections. In a specific embodiment, the inflammatory disorder which is prevented, treated, managed and/or ameliorated in accordance with the methods of the invention is undifferentiated spondyloarthropathy, undifferentiated arthropathy, arthritis, reactive arthritis, rheumatoid arthritis (juvenile and adult), osteoarthritis, psoriatic arthritis or other degenerative joint diseases. In another embodiment, the inflammatory disorder which is prevented, treated, managed and/or ameliorated in accordance with the methods of the invention is an inflammatory disorder characterized as a type 2-mediated inflammation. Type 2-mediated inflammation is characterized by eosinophilic and basophilic tissue infiltration and/or extensive mast cell degranulation, a process dependent on cross-linking of surface-bound IgE. In another embodiment, the inflammatory disorder which is prevented, treated, managed and/or ameliorated in accordance with the methods of the invention is asthma, Behcet's disease, pulmonary fibrosis, renal fibrosis, Gout or allergic disorders.

In a specific embodiment, the invention provides methods for preventing, treating, managing and/or ameliorating a condition associated with joint inflammation (e.g., undifferentiated spondyloarthropathy, undifferentiated arthropathy, arthritis, reactive arthritis, rheumatoid arthritis (juvenile and adult), osteoarthritis, psoriatic arthritis and/or other degenerative joint diseases), the methods comprising administering to a subject an effective amount of a theaflavin composition of the invention and an effective amount of one or more therapies (e.g., prophylactic or therapeutic agents) useful in preventing, treating, managing, and/or ameliorating such conditions or one or more symptoms thereof. Non-limiting examples of such therapies include glucosamine, methylsulfonylmethane, Bowellia extract, bromelain, tumeric extract, Feverfew, hops, phellodendron, devil's claw extract, gamma-linolenic acid, cat's claw, cis-9-cetylmyristoleate, chondroitin, collagen, fish oil, omega-3 fatty acids, ginger, ginkgo biloba, ginseng, gotu kola, grapeseed, gugulipid, melatonin, Noni, New Zealand green-lipped mussel, S-adenosyl-L-methionine, white willow bark, stinging nettle, deer antler velvet, Vitamin B3, Vitamin C, Vitamin E, boron, superoxide dismutase, back cohosh, cayenne, meadowsweet, alfalfa, yucca apple cider vinegar, cherry juice, hylaronic acid, celadrin, methotrexate, TNF-α antagonists (e.g., a soluble TNF-α receptor (e.g., entanercept (ENBREL™, Immunex)) and an antibody that specifically binds to TNF-α (e.g., infliximab (REMICADE™)), camphor, methyl/trolamine/salicylate/menthol, non-steroidal anti-inflammatory drugs (NSAIDs) (e.g., aspirin, ibuprofen, salicylates, acetominophen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™)), steroidal anti-inflammatory drugs (e.g., glucocorticoids, corticosteroids, dexamethasone, cortisone, hydrocortisone, prednisone (DELTASONE™), and prednisolone), and opioid medications (e.g., oxycodone and morphine).

In a preferred embodiment, the invention provides methods for preventing, treating, managing and/or ameliorating a condition associated with joint inflammation (e.g., undifferentiated spondyloarthropathy, undifferentiated arthropathy, arthritis, reactive arthritis, rheumatoid arthritis (juvenile and adult), osteoarthritis, psoriatic arthritis and/or other degenerative joint diseases), the methods comprising administering to a subject an effective amount of a theaflavin composition of the invention and an effective amount of a glucosamine composition. In accordance with this embodiment, the methods may further comprise the administration of an effective amount of one or more other therapies. In certain embodiments, such other therapies include methylsulfonylmethane, Bowellia extract, bromelain, tumeric extract, Feverfew, hops, phellodendron, devil's claw extract, gamma-linolenic acid, cat's claw, cis-9-cetylmyristoleate, chondroitin, collagen, fish oil, omega-3 fatty acids, ginger, ginkgo biloba, ginseng, gotu kola, grapeseed, gugulipid, melatonin, Noni, New Zealand green-lipped mussel, S-adenosyl-L-methionine, white willow bark, stinging nettle, deer antler velvet, Vitamin B3, Vitamin C, Vitamin E, boron, superoxide dismutase, back cohosh, cayenne, meadowsweet, alfalfa, yucca apple cider vinegar, cherry juice, hylaronic acid, celadrin, NSAIDs, methyl/trolamine/salicylate/menthol, and camphor.

In a specific embodiment, the invention provides methods for preventing, treating, managing and/or ameliorating a condition associated with joint inflammation (e.g., undifferentiated spondyloarthropathy, undifferentiated arthropathy, arthritis, reactive arthritis, rheumatoid arthritis (juvenile and adult), osteoarthritis, psoriatic arthritis and/or other degenerative joint diseases), the methods comprising administering to a subject an effective amount of a theaflavin composition of the invention, an effective amount of a glucosamine composition, and an effective amount of gotu kola. In accordance with this embodiment, the methods may further comprise the administration of an effective amount of superoxide dismutase. In another embodiment, the invention provides methods for preventing, treating, managing and/or ameliorating a condition associated with joint inflammation (e.g., undifferentiated spondyloarthropathy, undifferentiated arthropathy, arthritis, reactive arthritis, rheumatoid arthritis (juvenile and adult), osteoarthritis, psoriatic arthritis and/or other degenerative joint diseases), the methods comprising administering to a subject an effective amount of a theaflavin composition of the invention, an effective amount of a glucosamine composition, and an effective amount of superoxide dismutase.

In a specific embodiment, the invention provides methods for preventing, treating, managing and/or ameliorating a condition associated with joint inflammation (e.g., undifferentiated spondyloarthropathy, undifferentiated arthropathy, arthritis, reactive arthritis, rheumatoid arthritis (juvenile and adult), osteoarthritis, psoriatic arthritis and/or other degenerative joint diseases), the methods comprising administering to a subject an effective amount of a composition comprising glucosamine or a pharmaceutically acceptable salt, solvate or hydrate thereof (e.g., glucosamine sulfate, glucosamine hydrochloride, n-acetyl glucosamine) and one, two, three or all of the following or a pharmaceutically acceptable salt, solvate or hydrate thereof: theaflavin, theaflavin-3-gallate and/or theaflavin-3′-gallate, theaflavin-3,3′-digallate. In accordance with this embodiment, the methods may further comprise the administration of an effective amount of one or more other therapies.

In a specific embodiment, the invention provides methods for preventing, treating, managing and/or ameliorating asthma, the methods comprising administering to a subject an effective amount of a theaflavin composition of the invention and an effective amount of one or more therapies (e.g., prophylactic or therapeutic agents) useful in preventing, treating, managing, and/or ameliorating asthma or one or more symptoms thereof. Non-limiting examples of such therapies include bromelain, tumeric extract, Feverfew, hops, gamma-linolenic acid, cat's claw, cis-9-cetylmyristoleate, fish oil, omega-3 fatty acids, ginger, ginkgo biloba, ginseng, grapeseed, gugulipid, melatonin, Noni, New Zealand green-lipped mussel, white willow bark, stinging nettle, superoxide dismutase, black cohosh, meadowsweet, cherry juice, adrenergic stimulants (e.g., catecholamines (e.g., epinephrine, isoproterenol, and isoetharine), resorcinols (e.g., metaproterenol, terbutaline, and fenoterol), saligenins (e.g., salbutamol)), anticholinergics (e.g.,atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), corticosteroids (e.g., methlyprednisolone (MEDROL™), prednisone (PREDNISONE™ and DELTASONE™), and prednisolone (PRELONE™, PEDIAPRED™)), glucocorticoids (e.g., oral steroids or other systemic or oral steroids, and inhaled gucocoritcoids), other steroids, immunosuppressant agents (e.g., methotrexate and gold salts), leukotriene modifiers (e.g., montelukast (SINGULAIR™), zafirlukast (ACCOLATE™), and zileuton (ZYFLO™)), mast cell stabilizers (e.g., cromolyn sodium (INTAL™) and nedocromil sodium (TILADE™)), methylxanthines (e.g., theophylline (UNIPHYL™, THEO-DUR™, SLO-BID™, AND TEHO-42™)), and mucolytic agents (e.g., acetylcysteine)).

In a specific embodiment, the invention provides methods for preventing, treating, managing and/or ameliorating allergies, the methods comprising administering to a subject an effective amount of a theaflavin composition of the invention and an effective amount of one or more therapies (e.g., prophylactic or therapeutic agents) useful in preventing, treating, managing, and/or ameliorating allergies or one or more symptoms thereof. Non-limiting examples of therapies include bromelain, tumeric extract, Feverfew, hops, gamma-linolenic acid, cat's claw, cis-9-cetylmyristoleate, fish oil, omega-3 fatty acids, ginger, ginkgo biloba, ginseng, grapeseed, gugulipid, melatonin, Noni, New Zealand green-lipped mussel, white willow bark, stinging nettle, superoxide dismutase, black cohosh, meadowsweet, cherry juice, antimediator drugs (e.g., antihistamine, see Table 1), corticosteroids, decongestants, sympathomimetic drugs (e.g., α-adrenergic and β-adrenergic drugs), theophylline and its derivatives, glucocorticoids, and immunotherapies (e.g., repeated long-term injection of allergen, short course desensitization, and venom immunotherapy).

TABLE 1
H₁ ANTIHISTAMINES

| Chemical class and | |
|---|---|
| representative drugs | Usual daily dosage |
| Ethanolamine | |
| Diphehydramine | 25-50 mg every 4-6 hours |
| Clemastine | 0.34-2.68 mg every 12 hours |
| Ethylenediamine | |
| Tripelennamine | 25-50 mg every 4-6 hours |
| Alkylamine | |
| Brompheniramine | 4 mg every 4-6 hours; or 8-12 mg of SR |
| | form every 8-12 hour |
| Chlorpheniramine | 4 mg every 4-6 hours; or 8-12 mg of SR |
| | form every 8-12 hour |
| Triprolidine (1.25 mg/5 ml) | 2.5 mg every 4-6 hours |
| Phenothiazine | |
| Promethazine | 25 mg at bedtime |
| Piperazine | |
| Hydroxyzine | 25 mg every 6-8 hours |
| Piperidines | |
| Astemizole (nonsedating) | 10 mg/d |
| Azatadine | 1-2 mg every 12 hours |
| Cetirzine | 10 mg/d |
| Cyproheptadine | 4 mg every 6-8 hour |
| Fexofenadine (nonsedating) | 60 mg every 12 hours |
| Loratidine (nonsedating) | 10 mg every 24 hours |

In a specific embodiment, the invention provides methods for preventing, treating, managing and/or ameliorating COPD, the methods comprising administering to a subject an effective amount of a theaflavin composition of the invention and an effective amount of one or more therapies (e.g., prophylactic or therapeutic agents) useful in preventing, treating, managing, and/or ameliorating COPD or one or more symptoms thereof. Non-limiting examples of such therapies include bronchodilators (e.g., short-acting β₂-adrenergic agonist (e.g., albuterol, pirbuterol, terbutaline, and metaproterenol), long-acting β₂-adrenergic agonists (e.g., oral sustained-release albuterol and inhaled salmeterol), anticholinergics (e.g., ipratropium bromide), and theophylline and its derivatives (therapeutic range for theophylline is preferably 10-20 μg/mL)), glucocorticoids, exogenous α₁AT (e.g., α₁AT derived from pooled human plasma administered intravenously in a weekly dose of 60 mg/kg), oxygen, lung transplantation, lung volume reduction surgery, endotracheal intubation, ventilation support, yearly influenza vaccine and pneumococcal vaccination with 23-valent polysaccharide, exercise, and smoking cessation.

In a specific embodiment, the invention provides methods for preventing, treating, managing and/or ameliorating pulmonary fibrosis, the methods comprising administering to a subject an effective amount of a theaflavin composition of the invention and an effective amount of one or more therapies (e.g., prophylactic or therapeutic agents) useful in preventing, treating, managing, and/or ameliorating pulmonary fibrosis or one or more symptoms thereof. Non-limiting examples of such therapies include oxygen, corticosteroids (e.g., daily administration of prednisone beginning at 1-1.5 mg/kg/d (up to 100 mg/d) for six weeks and tapering slowly over 3-6 months to a minimum maintenance dose of 0.25 mg/kg/d), cytotoxic drugs (e.g., cyclophosphamide at 100-120 mg orally once daily and azathioprine at 3 mg/kg up to 200 mg orally once daily), bronchodilators (e.g., short- and long-acting β₂-adrenergic agonists, anticholinergics, and theophylline and its derivatives), and antihistamines (e.g., diphenhydramine and doxylamine).

The present invention also provides methods for preventing, managing, treating and/or ameliorating a condition associated with inflammation (e.g., an inflammatory condition), the methods comprising administering to a subject in need thereof an effective amount of a composition comprising a theaflavin composition and one or more prophylactic or therapeutic agents other than such a theaflavin composition. In a specific embodiment, a composition comprises a theaflavin composition, and one or more natural products, phytochemicals and/or botanical extracts, other than such a theaflavin composition. In a preferred embodiment, a composition comprises a theaflavin composition, a glucosamine composition, and optionally one or more other therapies. Depending on the manner of use, the compositions described herein can be, but not limited to, a dietary supplement, a food additive, a pharmaceutical composition, or a cosmetic composition.

Dosage & Frequency of Administration

The amount of a composition described herein which will be effective in the prevention, treatment, management, and/or amelioration of a condition associated with inflammation (e.g., an inflammatory disorder), or one or more symptoms thereof will vary with the nature and severity of the disease or condition, and the route by which the active ingredient is administered. The frequency and dosage will also vary according to factors specific for each subject or patient depending on the specific therapy (e.g., therapeutic or prophylactic agents) administered, the severity of the condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (59th ed., 2005).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In a specific embodiment, about 1 microgram per kilogram to about 500 milligrams per kilogram, about 100 micrograms per kilogram to about 5 milligrams per kilogram, or about 1 microgram per kilogram to about 50 micrograms per kilogram of the active ingredient in a composition described herein is administered to a subject in accordance with the methods of the invention. In certain embodiments, the active ingredient is a theaflavin or the invention. In other embodiments, the active ingredient is glucosamine.

In general, the recommended daily dose range of a composition described herein for the conditions described herein lie within the range of from about 0.01 mg to about 3000 mg per day, given as a single once-a-day dose or preferably as divided doses throughout a day. In one embodiment, the daily dose is administered twice daily in equally divided doses. Specifically, a daily dose range should be from about 5 mg to about 750 mg per day, more specifically, between about 10 mg and about 500 mg per day. In managing the subject or patient, the therapy should be initiated at a lower dose, perhaps about 1 mg to about 100 mg, and increased if necessary up to about 200 mg to about 3000 mg per day as either a single dose or divided doses, depending on the subject or patient's global response. It may be necessary to use dosages of the active ingredient outside the ranges disclosed herein in some cases, as will be apparent to those of ordinary skill in the art. Furthermore, it is noted that the dietitian, clinician or treating physician will know how and when to interrupt, adjust, or terminate therapy in conjunction with individual patient response.

Different effective amounts may be applicable for different conditions, as will be readily known by those of ordinary skill in the art. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject or patient is administered multiple dosages of a compound of the invention, not all of the dosages need be the same. For example, the dosage administered to the subject or patient may be increased to improve the prophylactic or therapeutic effect of the compound or it may be decreased to reduce one or more side effects that a particular subject or patient is experiencing.

In a specific embodiment, the dosage of a composition described herein administered to prevent, treat, manage, or ameliorate a condition associated with inflammation (e.g., an inflammatory disorder), or one or more symptoms thereof in a patient is about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of a composition described herein administered to prevent, treat, manage, or ameliorate a condition associated with inflammation (e.g., an inflammatory disorder), or one or more symptoms thereof in a patient is a unit dose of 0.1 mg to 3000 mg, 100 mg to 2500 mg, 150 mg, 2000 mg, 200 mg to 1500 mg, 500 mg to 1000 mg, 200 mg to 500 mg, 0.1 mg to 25 mg, 1 mg to 50 mg, or 10 mg to 100 mg.

In a specific embodiment, the dosage of a theaflavin composition administered to prevent, treat, manage, or ameliorate a condition associated with inflammation (e.g., an inflammatory disorder), or one or more symptoms thereof in a patient is about 150 μg/kg, preferably about 250 μμg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of a theaflavin composition described herein administered to prevent, treat, manage, or ameliorate a condition associated with inflammation (e.g., an inflammatory disorder), or one or more symptoms thereof in a patient is a unit dose of 0.1 mg to 3000 mg, 100 mg to 2500 mg, 150 mg to 2000 mg, 200 mg to 1500 mg, 500 mg to 1000 mg, 200 mg to 500 mg, 0.1 mg to 25 mg, 1 mg to 50 mg, or 10 mg to 100 mg.

In a specific embodiment, the dosage of a glucosamine composition administered to prevent, treat, manage, or ameliorate a condition associated with inflammation (e.g., an inflammatory disorder), or one or more symptoms thereof in a patient is about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of a glucosamine composition described herein administered to prevent, treat, manage, or ameliorate a condition associated with inflammation (e.g., an inflammatory disorder), or one or more symptoms thereof in a patient is a unit dose of 0.1 mg to 3000 mg, 100 mg to 2500 mg, 150 mg to 2000 mg, 200 mg to 1500 mg, 500 mg to 1000 mg, 200 mg to 500 mg, 0.1 mg to 25 mg, 1 mg to 50 mg, or 10 mg to 100 mg.

The dosages of prophylactic or therapeutic agents which have been or are currently being used to prevent, treat, manage, or ameliorate a condition associated with inflammation (e.g., an inflammatory disorder), or one or more symptoms thereof can be used in the combination therapies of the invention. Preferably, dosages lower than those which have been or are currently being used to prevent, treat, manage, or ameliorate a condition associated with inflammation (e.g., an inflammatory disorder), or one or more symptoms thereof are used in the combination therapies of the invention. The recommended dosages of agents currently used for the prevention, treatment, management, or amelioration of a condition associated with inflammation (e.g., an inflammatory disorder), or one or more symptoms thereof can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 59th ed., 2005, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies (e.g., prophylactic or therapeutic agents) are administered within the same patent visit.

In certain embodiments, a theaflavin composition and one or more other the therapies (e.g., prophylactic or therapeutic agents) are cyclically administered. Cycling therapy involves the administration of a first therapy (e.g., a theaflavin composition) for a period of time, followed by the administration of a second therapy (e.g., a prophylactic or therapeutic agent) for a period of time, followed by the administration of a third therapy (e.g., a prophylactic or therapeutic agent) for a period of time and so forth, and repeating this sequential administration, i.e., the cycle in order to reduce the development of resistance to one of the agents, to avoid or reduce the side effects of one of the therapies, and/or to improve the efficacy of the therapies.

In certain embodiments, administration of the same composition may be repeated and the administrations may be separated by at least 4 hours, 8 hours, 12 hours, 1 day, 2 days, 3 days, 5 days, 10 days, 2 weeks, 15 days, 3 weeks, 30 days, 45 days, 6 weeks, 2 months, 75 days, 12 weeks, 3 months, or 6 months.

In a specific embodiment, the invention provides a method of preventing, treating, managing, or ameliorating a condition associated with inflammation (e.g., an inflammatory disorder), or one or more symptoms thereof, the methods comprising administering to a subject in need thereof a dose of approximately 10 mg to approximately 1500 mg, more preferably a dose of approximately 100 mg to approximately 500 mg, of a theaflavin composition, and a dose of approximately 250 mg to approximately 3,000 mg, more preferably a dose of approximately 1,500 mg to approximately 2,500 mg, of a glucosamine composition. In another embodiment, the theaflavin composition is a tablet comprising 100 mg of the theaflavins of the invention, and the glucosamine composition is a tablet comprising 500 mg of glucosamine, a pharmaceutically acceptable salt, solvate or hydrate thereof. In accordance with these embodiments, the theaflavin composition comprises approximately 2% to approximately 95% by weight of one or more of the theaflavins of the invention, and the glucosamine composition comprises approximately 15% to approximately 98% by weight of glucosamine, a pharmaceutically acceptable salt, solvate or hydrate thereof.

In certain embodiments, a dose of approximately 500 mg/day to approximately 1200 mg/day of a theaflavin composition and a dose of approximately 1000 mg/day to approximately 2000 mg/day of a glucosamine composition are administered to a subject in need thereof to treat, manage and/or prevent a condition associated with joint inflammation and/or joint discomfort. In other embodiments, a dose of approximately 800 mg/day (in some embodiments, approximately 850 mg/day, approximately 900 mg/day, approximately 950 mg/day, approximately 975 mg/day, approximately 1000 mg/day, or approximately 1100 mg/day) of a theaflavin composition and a dose of approximately 1200 mg/day (in some embodiments, approximately 1250 mg/day, approximately 1300 mg/day, approximately 1350 mg/day, approximately 1400 mg/day, approximately 1450 mg/day, approximately 1500 mg/day, approximately 1550 mg/day or approximately 1600 mg/day) of a glucosamine composition are administered to a subject in need thereof to treat, manage and/or prevent a condition associated with joint inflammation (e.g., osteoarthritis) and/or joint discomfort. In specific embodiments, a dose of approximately 975 mg/day of a theaflavin composition (e.g., a black tea theaflavin extract) and a dose of approximately 1500 mg/day of a glucosamine composition (e.g., glucosamine hydrochloride) are administered to a subject in need thereof to treat, manage and/or prevent a condition associated with joint inflammation or joint discomfort. In some embodiments, the dosage of the theaflavin composition and the dosage of the glucosamine composition are administered to the subject for 2 weeks, 3 weeks, 4 weeks, 5 weeks, 6 weeks, 8 weeks, 12 weeks or more. In accordance with these embodiments, the theaflavin composition comprises approximately 2% to approximately 95% by weight of one or more of the theaflavins of the invention, and the glucosamine composition comprises approximately 15% to approximately 98% by weight of glucosamine, a pharmaceutically acceptable salt, solvate or hydrate thereof.

In one embodiment, a dose of approximately 100 mg/day to approximately 350 mg/day of a theaflavin composition and a dose of approximately 1,500 to approximately 2,500 mg/day of a glucosamine composition are administered to a subject to treat, osteoarthritis or joint discomfort and pain associated with inflammation. In another embodiment, a dose of approximately 25 mg/day to approximately 150 mg/day of a theaflavin composition and a dose of approximately 1,500 to approximately 2,500 mg/day of a glucosamine composition are administered to a subject to maintain joint health. In accordance with these embodiments, the theaflavin composition comprises approximately 2% to approximately 95% by weight of one or more of the theaflavins of the invention, and the glucosamine composition comprises approximately 15% to approximately 98% by weight of glucosamine, a pharmaceutically acceptable salt, solvate or hydrate thereof.

In certain embodiments, high doses of a theaflavin composition (e.g., about 100 mg/day to about 500 mg/day) and doses of about 250 mg/day to about 1,500 mg/day of a glucosamine composition are administered to a subject with a condition associated with inflammation for a certain period of time (e.g., 2 days, 5 days, 7 days, 10 days, and 14 days) to address pain relief and then lower doses of the theaflavin composition (e.g., 25 mg/day to 350 mg/day) and the same or higher doses (e.g., about 1,500 mg/day to about 2,500 mg/day) of the glucosamine composition are administered to the subject to maintain pain relief, reduce in inflammation and build cartilage. In a specific embodiment, doses of 100 mg/day to 500 mg/day of a theaflavin composition and doses of 250 mg/day to 1,500 mg/day of a glucosamine composition are administered to a subject with a condition associated with inflammation (e.g., an inflammatory disorder) for the first 5, 7 or 14 days of therapy, and then starting on the sixth, eighth, or fifteenth day of therapy, doses of 25 mg/kg to 350 mg/kg of the theaflavin composition and doses of 1,500 mg/kg to 2,500 mg/kg of the glucosamine composition are administered to the subject. In accordance with these embodiments, the theaflavin composition comprises approximately 2% to approximately 95% by weight of one or more of the theaflavins of the invention, and the glucosamine composition comprises approximately 15% to approximately 98% by weight of glucosamine, a pharmaceutically acceptable salt, solvate or hydrate thereof.

Biological Assays

Several aspects of the compositions or combination therapies described herein are preferably tested in vitro, in a cell culture system, and/or in an animal model organism, such as a rodent animal model system, for the desired activity prior to use in humans. For example, assays which can be used to determine whether administration of a specific composition ora specific combination of therapies is indicated, include cell culture assays in which a patient tissue sample is grown in culture, and exposed to or otherwise contacted with a composition, and the effect of such composition upon the tissue sample is observed. The tissue sample can be obtained by biopsy from the patient. This test allows the identification of the therapeutically most effective therapy for each individual patient. In various specific embodiments, in vitro assays can be carried out with representative cells of cell types involved in a condition associated with inflammation (e.g., immune cells), to determine if a composition has a desired effect upon such cell types. As an alternative to the use of tissue, tissue samples, established cell lines can be used in in vitro assays.

Compositions or combination therapies can be assayed for their ability to modulate the activation of various types of immune cells (including T cells, B cells, NK cells, macrophages, and dendritic cells). Activation of immune cells can be determined by measuring, e.g., changes in the level of expression and/or phospharylation of cytokines, and/or cell surface markers. Techniques known to those of skill in the art, including, but not limited to, immunoprecipitation followed by Western blot analysis, ELISAs, flow cytometry, Northern blot analysis, RT-PCR, real time PCR and microarray can be used to measure the expression of cytokines and cell surface markers indicative of activation of the immune cell.

Compositions or combination therapies can be assayed for their ability to induce the expression and/or activation of a gene product (e.g., cellular protein or RNA) and/or to induce signal transduction in cells (e.g., primary cells or established cell lines), including for example, immune cells and endothelial cells. The induction of the expression or activation of a gene product or the induction of signal transduction pathways in cells can be assayed by techniques known to those of skill in the art including, e.g., ELISAs, flow cytometry, Northern blot analysis, Western blot analysis, RT-PCR, kinase assays and electrophoretic mobility shift assays. Compositions or combination therapies can also be assayed for their ability to modulate cellular proliferation. Techniques known to those in art, including, but not limited to, ³H-thymidine incorporation, trypan blue cell counts, and fluorescence activated cell sorting (“FACS”) analysis. Compositions or combination therapies can also be assayed for their ability to induce cytolysis. Cytolysis can be assessed by techniques known to those in art, including, but not limited to, ⁵¹Cr-release assays.

In certain embodiments, compositions or combination therapies of the invention are assayed for their ability to induce NF-κB activation. In accordance with this embodiment, the phosphyloration of, e.g., I kappa B can be measured by immunoprecipitation followed by Western blot analysis. In another embodiment, compositions or combination therapies of the invention are assayed for their ability to induce nuclear translocation and the DNA binding activity of NF-κB by, e.g., an electromobility shift assay (EMSA). In yet another embodiment, compositions or combination therapies of the invention are assayed for their ability to alter the expression profile of a gene whose expression is regulated by NF-κB (e.g., IL-2, IL-6, IL-8, IL-12A, IL-12B, IRF-1, TNF-α, TNF-β, GM-CSF, G-CSF, IFNα1, IFN-β1, angiotensinogen, C3, ICAM1, ICAM2, ICAM3, ICAM4, ICAM5, E-selectin, L-selectin, P-selectin, VCMA-1, etc.) by, e.g., ELISA, Western blot, Northern blot, RT-PCR, etc. In yet another embodiment, compositions or combination therapies of the invention are assayed for their ability to induce Cox-2 gene expression by, e.g., Northern blot, RT-PCR, Western blot, ELISA, etc.

Compositions or combination therapies can also be assayed for their ability to inhibit cell migration or cell adhesion using techniques well-known to one of skill in the art or described herein. The compositions or combination therapies can also be assayed for their ability to induce cell cycle arrest or apoptosis.

Compositions or combination therapies can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the compositions or combination therapies described herein are tested in a mouse model system. Such model systems are widely used and well-known to the skilled artisan. Compositions or combination therapies can be administered repeatedly. Several aspects of the procedure may vary including, but not limited to, temporal regime for administration of the compositions or combination therapies.

The anti-inflammatory activity of the compositions or combination therapies described herein can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993). Experimental and spontaneous animal models of inflammatory arthritis and autoimmune rheumatic diseases can also be used to assess the anti-inflammatory activity of the compositions described herein. The following are some assays provided as examples and not by limitation.

The principle animal models for arthritis or inflammatory disease known in the art and widely used include: adjuvant-induced arthritis rat models, collagen-induced arthritis rat and mouse models and antigen induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993), incorporated herein by reference in its entirety.

The anti-inflammatory activity of the compositions or combination therapies described herein can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hamra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the compositions or combination therapies described herein can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of a composition described herein is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the compositions or combination therapies described herein. Alternatively, the efficacy of the compositions or combination therapies described herein can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

Additionally, animal models for inflammatory bowel disease can also be used to assess the efficacy of the compositions or combination therapies described herein (Kim et al., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of the compositions or combination therapies described herein. An example of one such model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747).

Animal models for psoriasis can also be used to assess the efficacy of the compositions or combination therapies described herein. Animal models for psoriasis have been developed (see, e.g., Schon, 1999, J. Invest. Dermatol. 112:405-410) and known in the art.

Further, any assays known to those skilled in the art can be used to evaluate the prophylactic and/or therapeutic utility of the compositions or combination therapies described herein for a condition associated with inflammation.

The toxicity and/or efficacy of the compositions or combination therapies described herein can be determined by standard pharmaceutical procedures in cell cultures or experimental animals, e.g., for determining the LD ₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions or combination therapies described herein that exhibit large therapeutic indices are preferred. While compositions or combination therapies described herein that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage of the compositions or combination therapies described herein for use in humans. The dosage of such agents lies preferably within a range of circulating rdnikadratiblinhatirielude the ED₅₀ with little or no toxicity. The dosage may vary within this range depending upon the dosage form employed and the route of administration utilized. For any agent used in the method of the invention, the prophylactically or therapeutically effective dose can be estimated initially from cell culture assays. A dose may be formulated in animal models to achieve a circulating plasma concentration range that includes the IC₅₀ (i.e., the concentration of the a composition that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing, managing and/or treating an inflammatory disorder may be demonstrated, e.g., by detecting the ability of the compositions or combination therapies described herein to reduce inflammation of a joint, organ or tissue, reduce one or more symptoms associated with an inflammatory disorder, to decrease T cell activation, to decrease T cell proliferation, to modulate one or more cytokine profiles, to reduce cytokine production, and/or to improve quality of life. Changes in inflammatory disease activity may be assessed through tender and swollen joint counts, patient and physician global scores for pain and disease activity (e.g., the Investigator Global Assessments of Disease Status (IGADS)), and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ) and the Western Ontario and McMaster Universities Osteoarthritis Index (WOMAC). Quality of life changes in humans with an inflammatory disorder may be assessed with the SF-36.

In certain embodiments, the prophylactic and/or therapeutic effect of compositions or combination therapies described herein is evaluated in the human endotoxemia model. See, e.g., van Eijk et al., Crit. Care 9(2): R157-164 (2005); Copeland et al., Clin. Diagn. Lab. Immunol. 12 (1): 60-67 (2005); De et al, J. Immunol. 175(9): 6155-6162 (2005); Qian et al., Proteomics 5(2): 572-584 (2005); and Steiner et al., Circulation 111(14): 1841-1846 (2005) for a description of the human endotoxin model. In a specific embodiment, the human endotoxemia model is used to evaluate the prophylactic and/or therapeutic effect (e.g., the anti-inflammatory effect) of the combination of a theaflavin composition (e.g., a dose of approximately 800 to approximately 1000 mg/day of a theaflavin composition, such as a theaflavin black tea extract) and a glucosamine composition (e.g., a dose of approximately 1200 to approximately 2000 mg/day of a glucosamine composition, such as glucosamine hydrochloride).

In certain embodiments, the prophylactic and/or therapeutic effect of the compositions or combination therapies described herein is evaluated in a delayed-onset muscle soreness model. See, e.g., Rahnama et al., Clin. J. Sport Med. 13(4): 200-208 (2003) and Goldstein, J. of Undergraduate Research: University of Florida, 2(5) (2001) for description of the delayed-onset muscle soreness model. In a specific embodiment, the delayed-onset muscle soreness model is used to evaluate the prophylactic and/or therapeutic effect (e.g., the anti-inflammatory effect) of the combination of a theaflavin composition (e.g., a dose of approximately 800 mg/day to approximately 1000 mg/day of a theaflavin composition such as theaflavin black tea extract) and a glucosamine composition (e.g., a dose of approximately 1200 mg/day to approximately 2000 mg/day of a glucosamine composition, such as glucosamine hydrochloride).

In certain embodiments, the prophylactic and/or therapeutic effect of the compositions or combination therapies described herein is evaluated in the molar extraction model. See, e.g., Anthonsen et al., JBC 276(32):30527-30536 (2001); Barosi et al., J. Invest. Dermatol. 119(5): 1020-1026 (2002); Gordon et al., Anesth. Analg. 95(5): 1351-1357 (2002); and Kim et al, J. Pain 5(7): 377-384 (2004) for a description of the molar extraction model. In a specific embodiment, the molar extraction model is used to evaluate the prophylactic and/or therapeutic effect (e.g., the anti-inflammatory effect) of the combination of a theaflavin composition (e.g., a dose of approximately 800 mg/day to approximately 1000 mg/day of a theaflavin composition such as a theaflavin black tea extract) and a glucosamine composition (e.g., a dose of approximately 1200 mg/day to approximately 2000 mg/day of a glucosamine composition, such as glucosamine hydrochloride).

Article of Manufacture

The invention encompasses an article of manufacture that can simplify the administration of a composition described herein to a subject. A typical article of manufacture of the invention comprises a unit dosage form of a composition described herein. In one embodiment, the unit dosage form is a container, preferably a sterile container, containing and effective amount of a composition described herein. The article of manufacture can further comprise a label or printed instructions regarding the use of the composition or other informational material that advises the dietitian, physician, technician, consumer, subject, or patient on how to appropriately prevent or treat the condition in question. In other words, the article of manufacture includes instruction means indicating or suggesting a dosing regimen including, but not limited to, actual doses, monitoring procedures, and other monitoring information. The article of manufacture can also further comprise a unit dosage form of another prophylactic or therapeutic agent, for example, a container containing an effective amount of another prophylactic or therapeutic agent.

In a specific embodiment, the article of manufacture comprises a container containing an effective amount of a theaflavin composition and a container containing an effective amount of another propylactic or therapeutic agent and a pharmaceutically acceptable carrier or excipient. Examples of other prophylactic or therapeutic agents include, but are not limited to, those listed above (See, e.g., Section 4.2 and 4.3, supra). In a particular embodiment, the article of manufacture comprises a unit dosage form of a theaflavin composition in one container and a unit dosage form of a glucosamine composition in a container. In a specific embodiment, the article of manufacture comprises a container containing an effective amount of a theaflavin composition, a container containing an effective amount of glucosamine composition, and optionally a container comprising another prophylactic or therapeutic agent. As with any pharmaceutical product and dietary supplement, the packaging material and container included in the article of manufacture are designed to protect the stability of the product during storage and shipment.

In another embodiment, the article of manufacture comprises a container containing an effective amount of a composition comprising a theaflavin(s) of the invention, glucosamine or a pharmaceutically acceptable salt, solvate or hydrate thereof, a pharmaceutically acceptable carrier or excipient, and optionally one or more other prophylactic or therapeutic agents. Examples of other prophylactic or therapeutic agents include, but are not limited to, those listed above (See, e.g., Section 4.2, supra). As with any pharmaceutical product and dietary supplement, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, drip bags, patches, and inhalers.

Articles of manufacture of the invention can further comprise pharmaceutically acceptable vehicles or consumable vehicles that can be used to administer one or more active ingredients. For example, if an active ingredient is provided in a solid form that must be reconstituted for parenteral or oral/enteral administration, the article of manufacture can comprise a sealed container of a suitable vehicle in which the active ingredient can be dissolved. For parenteral administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLE

This example describes a randomized, double-blind, placebo-controlled study to evaluate the efficacy, safety, and tolerability of the combination of glucosamine and black tea theaflavins extract in subjects with osteoarthritis of the knee

| | |
|---|---|
| Indication: | Analgesic to manage osteoarthritic pain |
| Objectives: | To assess the efficacy of the following in subjects with |
| | osteoarthritis (OA) of the knee: |
| | 1. black tea theaflavins extract (WG0401) alone (975 mg) |
| | 2. glucosamine hydrochloride alone (1500 mg) |
| | 3. glucosamine hydrochloride (1500 mg) and black tea |
| | theaflavins extract (WG0401, 975 mg) combined |
| | 4. placebo |
| | To determine the safety and tolerability of black tea theaflavins |
| | assessed by spontaneous adverse event reporting and clinical |
| | laboratory measurements. |
| Number of Subjects: | 80 human subjects (4 arms of 20 to be recruited) |
| Study Population: | Male and female adult (≧40 years) subjects with osteoarthritis of |
| | the knee. |
| Study Design: | This is a three-month, randomized, double-blinded, parallel- |
| | group, placebo-controlled study. |
| Treatments: | Participants are randomly assigned to receive one of four |
| | treatments (as described above in objectives), daily for 12 weeks. |
| Primary Endpoint: | Improvement in WOMAC Index scores (3 subscales - pain, |
| | stiffness and physical function) are used to assess efficacy. |
| Secondary Endpoints: | Patient's assessment of pain |
| | Physician assessment of pain (Investigator Global Assessment |
| | of Disease Status—IGADS) |
| | GAITRite measurements |
| | Safety and tolerability assessed by spontaneous adverse event |
| | reporting and clinical laboratory measurements. |
| Safety Measurement/ | Clinical laboratory measurements, vital signs, and self-reported |
| Parameters: | signs/symptoms consistent with adverse events are evaluated. |
| | Biomarkers of inflammation are also assessed. |
| Statistical Methods: | Treatment arm comparisons of the primary endpoint are made |
| | using a two-group repeated measures analysis of variance. |
| | Secondary endpoints and other collected clinical and laboratory |
| | data are analyzed using appropriate parametric and non- |
| | parametric equivalent statistics. |

Study Schedule/Flowchart:

| | Day 0 | Day 1 | Weeks | | |
|---|---|---|---|---|---|
| | Screening | Baseline | 4 | 8 | 12 |
| Combination/WG0401/Placebo | | X | X | X | X |
| Informed Consent | X | | | | |
| Medical History | X | | | | |
| Complete Physical Exam | X | | | | |
| Limited Physical Exam | | X | X | X | X |
| Vital Signs | X | X | X | X | X |
| Patient's Assessment of Pain | X | X | X | X | X |
| Physician Assessment of Pain | X | X | X | X | X |
| WOMAC* Visual Analog Scale | X | X | X | X | X |
| GAITRite Measurements | X | X | X | X | X |
| Adverse Event Reporting | | X | X | X | X |

*Western Ontario and McMaster Universities Osteoarthritis Index

Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A method for preventing a condition associated with inflammation, the method comprising administering to a human subject a prophylactically effective amount of a theaflavin composition and a prophylactically effective amount of a therapy other than a theaflavin composition.

2. A method for treating a condition associated with inflammation, the method comprising administering to a human subject in need thereof a therapeutically effective amount of a theaflavin composition and a therapeutically effective amount of a therapy other than a theaflavin composition.

3. (canceled)

4. The method of claim 1 or 2, wherein the disorder is an inflammatory disorder., asthma, encephilitis, inflammatory bowel disease, chronic obstructive pulmonary disease (COPD), allergic disorders, septic shock, fibrosis, undifferentiated spondyloarthropathy, undifferentiated arthropathy, arthritis, juvenile arthritis, psoriatic arthritis, rheumatoid arthritis, psoriasis, or inflammatory osteolysis.

5. A method of treating or managing condition associated with joint inflammation, the method comprising administering to a human subject in need thereof an effective amount of a theaflavin composition and an effective amount of a glucosamine composition.

6. A method of treating or managing condition associated with joint inflammation, the method comprising administering to a human subject in need thereof an effective amount of a composition comprising theaflavin, theaflavin-3-gallate, theaflavin-3′-gallate, theaflavin-3,3′-digallate, or a pharmaceutically acceptable salt, solvate or hydrate thereof and glucosamine or a pharmaceutically acceptable salt, solvate or hydrate thereof.

7. The method of claim 1, 2 or 5, wherein the theaflavin composition comprises theaflavin, theaflavin-3-gallate, theaflavin-3′-gallate, theaflavin-3,3′-digallate, or a pharmaceutically acceptable salt, solvate or hydrate thereof.

8. The method of claim 1, 2 or 5, wherein the theaflavin composition comprises two or more of the following theaflavins: theaflavin, theaflavin-3-gallate, theaflavin-3′-gallate, and theaflavin-3,3′-digallate, or a pharmaceutically acceptable salts, solvates or hydrates thereof.

9. The method of claim 1, 2 or 5, wherein theaflavin composition comprises theaflavin, theaflavin-3-gallate, theaflavin-3′-gallate, and theaflavin-3,3′-digallate.

10. The method of claim 1, 2 or 5, wherein the theaflavin composition is a black tea extract.

11. The method of claim 5, wherein the glucosamine composition comprises glucosamine sulfate, glucosamine hydrochloride, or n-acetyl glucosamine.

12. The method of claim 1 or 2, wherein the other therapy is glucosamine, methylsulfonylmethane, Bowellia extract, bromelain, tumeric extract, Feverfew, hops, phellodendron, devil's claw extract, gamma-linolenic acid, cat's claw, cis-9-cetylmyristoleate, chondroitin, collagen, fish oil, omega-3 fatty acids, ginger, ginkgo biloba, ginseng, gotu kola, grapeseed, gugulipid, melatonin, Noni, New Zealand green-lipped mussel, S-adenosyl-L-methionine, white willow bark, stinging nettle, deer antler velvet, Vitamin B3, Vitamin C, Vitamin E, boron, superoxide dismutase, back cohosh, cayenne, meadowsweet, alfalfa, yucca apple cider vinegar, cherry juice, hylaronic acid, celadrin, methotrexate, a TNF-α antagonist, camphor, methyl/trolamine/salicylate/menthol, a non-steroidal anti-inflammatory drug (NSAID), or an opioid medication.

13. The method of claim 5 or 6 further comprising administering at least one other therapy.

14. The method of claim 13, wherein the other therapy is methylsulfonylmethane, Bowellia extract, bromelain, tumeric extract, Feverfew, hops, phellodendron, devil's claw extract, gamma-linolenic acid, cat's claw, cis-9-cetylmyristoleate, chondroitin, collagen, fish oil, omega-3 fatty acids, ginger, ginkgo biloba, ginseng, gotu kola, grapeseed, gugulipid, melatonin, Noni, New Zealand green-lipped mussel, S-adenosyl-L-methionine, white willow bark, stinging nettle, deer antler velvet, Vitamin B3, Vitamin C, Vitamin E, boron, superoxide dismutase, back cohosh, cayenne, meadowsweet, alfalfa, yucca apple cider vinegar, cherry juice, hylaronic acid, celadrin, methotrexate, a TNF-α antagonist, camphor, methyl/trolamine/salicylate/menthol, a non-steroidal anti-inflammatory drug (NSAID), or an opioid medication.

15. The method of claim 1, 2 or 5, wherein the theaflavin composition is a nutraceutical composition, a pharmaceutical composition, or a dietary supplement.

16. The method of claim 5, wherein the glucosamine composition is a nutraceutical composition, a pharmaceutical composition, or a dietary supplement.

17.-20. (canceled)

21. The method of claim 5 or 6, wherein the condition is undifferentiated spondyloarthropathy, undifferentiated arthropathy, arthritis, reactive arthritis, rheumatoid arthritis, osteoarthritis or psoriatic arthritis.

22. The method of claim 1, 2 or 5, wherein the effective amount of the theaflavin composition is a dosage of approximately 10 mg to approximately 1500 mg.

23. The method of claim 5, wherein the effective amount of the glucosamine composition is a dosage of approximately 250 mg to approximately 3,000 mg.

24. The method of claim 6, wherein the glucosamine is glucosamine sulfate, glucosamine hydrochloride or n-acetyl glucosamine.

25. The method of claim 6, wherein the composition is a pharmaceutical composition, a pharmaceutical composition, or a dietary supplement.

26.-27. (canceled)
